(19) United States
(12) Patent Application Publication
(10) Pub. No.: US 2005/0113419 A1
(43) Pub. Date: May 26, 2005

(54) Therapeutic compounds for treating dyslipidemic conditions

(76) Inventors: Shaei Y. Huang, Lansdale, PA (US); Alan D. Adams, Cranford, NJ (US)

Correspondence Address: MERCK AND CO., INC, P O BOX 2000, RAHWAY, NJ, 07065-0907, US

(21) Appl. No.: 10/973,715

(22) Filed: Oct. 26, 2004

Related U.S. Application Data
(60) Provisional application No. 60/516,433, filed on Oct. 31, 2003.

(51) Int. Cl. C07D 417/02; C07D 413/02; A61K 31/4439; A61K 31/427; A61K 31/42

(52) U.S. Cl. 514/338; 514/379; 514/365; 514/374; 546/272.1; 548/181; 548/215; 548/241

(57) ABSTRACT

The present invention relates to novel LXR ligands of Formula I 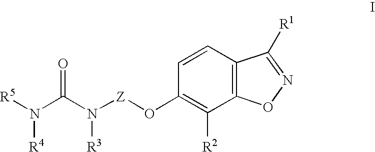 and the pharmaceutically acceptable salts, esters and tautomers thereof, which are useful in the treatment of dyslipidemic conditions, particularly depressed levels of HDL cholesterol.

### BACKGROUND OF THE INVENTION

Recent publications in Nature Genetics, August, 1999 (Young et al., page 316; Bodzioch et al., page 347; Brooks-Wilson et al., page 335, and Rust et al., page 352) showed that humans with mutations in the gene ABCA1 (also previously known in the art as ABC1) have low levels of high density lipoprotein (HDL). Low HDL levels are a risk factor for atherosclerosis, myocardial infarction and related conditions such as ischemic stroke. Therefore, increasing the expression of the ABCA1 gene would be expected to increase HDL levels and decrease the occurrence of atherosclerosis, myocardial infarction and related conditions such as ischemic stroke. It has been reported that expression of the ABCA1 gene is increased by cholesterol loading of cells (Langmann et al., Biochem. Biophys. Res. Comm., 257, 29-33 (1999)). LXRα is a nuclear receptor that is required for the induction of cholesterol 7α-hydroxylase in mouse liver following cholesterol feeding (Peet et al., Cell, 93, 693-704 (1998)). LXRα (NR1H3) (for a unified system of nomenclature for the nuclear receptor superfamily see Cell 97, 161-163, 1999) and LXRβ (NR1H2) are activated by 22-(R)-hydroxycholesterol and other oxysterols (Janowski et al. Proc. Natl. Acad. Sci USA , 96, 266-271 (1999), Spencer et al. J. Med. Chem., 44, 886-897, (2001)). Some non-steroidal small molecule agonists of LXRα and LXRβ have been reported to affect circulating HDL levels, cholesterol absorption, reverse cholesterol transport and ABCA1 expression in vivo (Collins et al J. Med. Chem 45, 1963-1966, (2002); Schultz, et al. Genes & Devel. 14, 2831-2838, (2000), Repa et al. Science, 289, 1524-1529, (2000)). A small molecule agonist of LXR has also been demonstrated to inhibit the development of atherosclerosis in a rodent model (Joseph et al. PNAS 99, 7604-7609, (2002)). It has been found that LXRα and/or LXRβ cause the induction or regulation of ABCA1 expression, and that small molecule ligands of LXR are useful as drugs to increase the expression of ABCA1, increase levels of HDL and thereby decrease the risk of atherosclerosis, myocardial infarction and related conditions such as peripheral vascular disease and ischemic stroke.

The various dyslipidemic conditions, which are risk factors for atherosclerosis, are currently treated with several different classes of drugs, such as statins which are HMG-CoA reductase inhibitors, bile acid sequestrants (e.g., cholestyramine and colestipol), nicotinic acid (niacin), and fibrates. However, except for niacin, most of these treatments do not raise HDL as their primary effect. With favorable outcomes in many human studies, the statin class of drugs is used to modulate LDL and, to a lesser extent, HDL and triglycerides. Conditions principally characterized by elevated plasma triglycerides and low HDL are frequently treated with drugs belonging to the fibrate class. The fibrates are PPAR alpha agonists that lower triglycerides and raise HDL in many instances. There are no currently marketed drugs whose principal actions are mediated by LXR.

We have now discovered a new class of small molecules which are LXR ligands, i.e., LXRα and/or LXRβ ligands, and are therefore expected to be useful for modulation of HDL levels, ABCA1 gene expression and reverse cholesterol transport. The instant compounds have been shown to raise plasma levels of HDL in animal models and to increase cholesterol efflux from cells in vitro. These biological activities are critical for reverse cholesterol transport.

The novel compounds of this invention are intended as a treatment for dyslipidemias, especially low plasma HDL cholesterol levels, as well as for treatment and/or prevention of lipid accumulation in atherosclerotic plaques, which is an underlying cause or aggravating factor in atherosclerosis.

### SUMMARY OF THE INVENTION

Compounds of Formula I below are novel LXR ligands which are useful in the treatment of dyslipidemic conditions including below-desirable levels of HDL cholesterol:

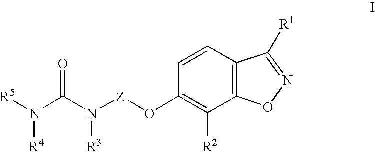

One object of the instant invention is to provide a method for treating depressed plasma HDL cholesterol levels comprising administering a therapeutically effective amount of a compound of Formula I to a patient in need of such treatment.

Another object is to provide a method for preventing or treating dyslipidemic conditions comprising administering a prophylactically or therapeutically effective amount, as appropriate, of a compound of Formula I to a patient in need of such treatment.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a patient who is at risk of developing atherosclerosis or who already has atherosclerotic disease. The method of this invention also serves to remove cholesterol from tissue deposits such as xanthomas and atherosclerotic lesions by hastening the efflux of cholesterol from cells in those lesions.

Another object of the present invention is the use of the compounds of the present invention for the manufacture of a medicament useful in treating, preventing or reducing the risk of developing these conditions.

Other objects of this invention are to provide processes for making the compounds of Formula I and to provide novel pharmaceutical compositions comprising these compounds. Additional objects will be evident from the following detailed description.

### DETAILED DESCRIPTION OF THE INVENTION

The novel LXR ligands of the instant invention are compounds of Formula I 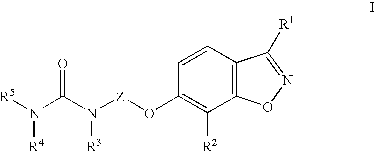
and the pharmaceutically acceptable salts, esters and tautomets thereof, wherein
R¹ is selected from the group consisting of:
   (a) —CF₃,
   (b) —CH₂C(CH₃)₃,
   (c) phenyl, unsubstituted, mono- or polysubstituted with halo,
   (d) —Cl₁₋₆ alkyl, and
   (e) —Cl₁₋₂ alkyl-phenyl;
R² is selected from the group consisting of:
   (a) —C₁₋₆ alkyl,
   (b) —CO₂R⁶,
   (c) —CR⁶R⁷—O—R⁸,
   (d) —CR⁶R⁷—S—R⁸, and
   (e) COR⁶;
R³ is —C₁₋₆alkyl;
R⁴ is —H or —C₁₋₆alkyl;
R⁵ is selected from the group consisting of:
   (a) —H,
   (b) —C₁₋₆ alkyl, unsubstituted or monosubstituted with —CO₂R⁶,
   (c) phenyl, unsubstituted or monosubstituted with —CO₂R⁶,
   (d) tetrazolyl,
   (e) oxazolyl, unsubstituted, mono- or polysubstituted with a substituent independently selected at each occurrence from the group consisting of halo, —C₁₋₆alkyl and —CO₂R⁶,
   (f) thiazolyl, unsubstituted, mono- or polysubstituted with a substituent independently selected at each occurrence from the group consisting of halo, —C₁₋₆alkyl and —CO₂R⁶,
   (g) pyridyl, unsubstituted, mono- or polysubstituted with a substituent independently selected at each occurrence from the group consisting of halo, —C₁₋₆alkyl and —CO₂R⁶,
   (h) pyrimidinyl, unsubstituted, mono- or polysubstituted with a substituent independently selected at each occurrence from the group consisting of halo, —C₁₋₆alkyl and —CO₂R⁶,
   (i) pyrazinyl, unsubstituted, mono- or polysubstituted with a substituent independently selected at each occurrence from the group consisting of halo, —C₁₋₆alkyl and —CO₂R⁶, and
   (j) N-oxo-pyridyl, unsubstituted, mono- or polysubstituted with a substituent independently selected at each occurrence from the group consisting of halo, —C₁₋₆alkyl and —CO₂R⁶;
R⁶, R⁷ and R⁸ are independently selected at each occurrence from the group consisting of —H, phenyl, and —C₁₋₆ alkyl; and
Z is —C₁₋₆ alkanediyl-.

In one embodiment of the present invention are compounds of Formula I wherein R¹ is —CF₃; R² is —C₁₋₆ alkyl, and more particularly n-propyl; R³ is C₁₋₆alkyl, particularly selected from methyl and ethyl, and more particularly methyl; R⁴ is —H or methyl, and particularly —H; and Z is -n-propanediyl-.

In a second embodiment of the present invention are those compounds of Formula I wherein R⁵ is —C₁₋₆ alkyl, unsubstituted, mono- or polysubstituted with CO₂R⁶. In a class of the second embodiment are those compounds of Formula I wherein R⁵ is —C₁₋₃ alkyl, unsubstituted, mono- or polysubstituted with CO₂R⁶; R¹ is —CF³; R² is —C₁₋₆ alkyl, and more particularly n-propyl; R³ is C₁₋₆alkyl, particularly selected from methyl and ethyl, and more particularly methyl; R⁴ is —H or methyl, and particularly —H; and Z is -n-propanediyl-. Examples of compounds within this embodiment and class include:
(1) N,N′-dimethyl-N-(3-{[7-propyl-3-(trifluoromethyl)-1,2-benzisoxazol-6-yl]oxy}propyl)urea,
(2) N′-ethyl-N-methyl-N-(3-{[7-propyl-3-(trifluoromethyl)-1,2-benzisoxazol-6-yl]oxy}propyl)urea,
(3) N′-propyl-N-methyl-N-(3-{[7-propyl-3-(trifluoromethyl)-1,2-benzisoxazol-6-yl]oxy}propyl)urea,
(4) N′-isopropyl-N-methyl-N-(3-{[7-propyl-3-(trifluoromethyl)-1,2-benzisoxazol-6-yl]oxy}propyl)urea,
(5) N′-(2-carbethoxyethyl)-N-methyl-N-(3-{[7-propyl-3-(trifluoromethyl)-1,2-benzisoxazol-6-yl]oxy}propyl)urea,
(6) N′-methyl-N-ethyl-N-(3-{[7-propyl-3-(trifluoromethyl)-1,2-benzisoxazol-6-yl]oxy}propyl)urea,
(7) N′,N-diethyl-N-(3-{[7-propyl-3-(trifluoromethyl)-1,2-benzisoxazol-6-yl]oxy}propyl)urea,
(8) N′-propyl-N-ethyl-N-(3-{[7-propyl-3-(trifluoromethyl)-1,2-benzisoxazol-6-yl]oxy}propyl)urea,
(9) N′-isopropyl-N-ethyl-N-(3-{[7-propyl-3-(trifluoromethyl)-1,2-benzisoxazol-6-yl]oxy}propyl)urea,
(10) N′-(2-carboxyethyl)-N-ethyl-N-(3-{[7-propyl-3-(trifluoromethyl)-1,2-benzisoxazol-6-yl]oxy}propyl)urea,
(11) N′-carboxymethyl-N-ethyl-N-(3-{[7-propyl-3-(trifluoromethyl)-1,2-benzisoxazol-6-yl]oxy}propyl)urea,
(12) N′-(1(S)-carboxyethyl)-N-methyl-N-(3-{[7-propyl-3-(trifluoromethyl)-1,2-benzisoxazol-6-yl]oxy}propyl)urea,
(13) rac-N′-methyl-N′-(1-carboxyethyl)-N-methyl-N-(3-{[7-propyl-3-(trifluoromethyl)-1,2-benzisoxazol-6-yl]oxy}propyl)urea,
(14) rac-N′-(1-carboxyethyl)-N-methyl-N-(3-{[7-propyl-3-(trifluoromethyl)-1,2-benzisoxazol-6-yl]oxy}propyl)urea,
(15) rac-N′-(2-carboxypropyl)-N-methyl-N-(3-{[7-propyl-3-(trifluoromethyl)-1,2-benzisoxazol-6-yl]oxy}propyl)urea,
(16) N′-ethyl-N′-methyl-N-methyl-N-(3-{[7-propyl-3-(trifluoromethyl)-1,2-benzisoxazol-6-yl]oxy}propyl)urea, and
(17) N′,N′-dimethyl-N-methyl-N-(3-{[7-propyl-3-(trifluoromethyl)-1,2-benzisoxazol-6-yl]oxy}propyl)urea;
   and the pharmaceutically acceptable salts, esters and tautomers thereof.

In a third embodiment are those compounds of Formula I wherein R5 is selected from pyridyl, N-oxo-pyridyl, pyrimidinyl, pyrazinyl and tetrazolyl wherein each of pyridyl, N-oxo-pyridyl, pyrimidinyl and pyrazinyl may be unsubstituted, mono- or polysubstituted with a substituent independently selected at each occurrence from the group consisting of halo, —C₁₋₆alkyl and —CO₂R⁶. In a class of the third embodiment are those compounds of Formula I wherein R¹ is —CF₃; R² is —C₁₋₆ alkyl, and more particularly n-propyl; R³ is C₁₋₆alkyl, particularly selected from methyl and ethyl, and more particularly methyl; R⁴ is —H or methyl, and particularly —H; and Z is -n-propanediyl-. Examples of compounds within this embodiment and class include:
(18) N′-(2-pyridyl)-N-methyl-N-(3-{[7-propyl-3-(trifluoromethyl)-1,2-benzisoxazol-6-yl]oxy}propyl)urea,
(19) N′-(3-pyridyl)-N-methyl-N-(3-{[7-propyl-3-(trifluoromethyl)-1,2-benzisoxazol-6-yl]oxy}propyl)urea,
(20) N′-(4-pyridyl)-N-methyl-N-(3-{[7-propyl-3-(trifluoromethyl)-1,2-benzisoxazol-6-yl]oxy}propyl)urea,
(21) N′-(2-pyridyl)-N′-methyl-N-methyl-N-(3-{[7-propyl-3-(trifluoromethyl)-1,2-benzisoxazol-6-yl]oxy}propyl)urea,
(22) N′-methyl-N′-(4-pyridyl)-N-methyl-N-(3-{[7-propyl-3-(trifluoromethyl)-1,2-benzisoxazol-6-yl]oxy}propyl)urea,
(23) N′-(6-methyl-2-pyridyl)-N-methyl-N-(3-{[7-propyl-3-(trifluoromethyl)-1,2-benzisoxazol-6-yl]oxy}propyl)urea,
(24) N′-(4,6-dimethyl-2-pyridyl)-N-methyl-N-(3-{[7-propyl-3-(trifluoromethyl)-1,2-benzisoxasol-6-yl]oxy}propyl)urea,
(25) N′-(4-methyl-2-pyridyl)-N-methyl-N-(3-{[7-propyl-3-(trifluoromethyl)-1,2-benzisoxazol-6-yl]oxy}propyl)urea,
(26) N′-(5-chloro-2-pyridyl)-N-methyl-N-(3-{[7-propyl-3-(trifluoromethyl)-1,2-benzisoxazol-6-yl]oxy}propyl)urea,
(27) N′-(5-methyl-2-pyridyl)-N-methyl-N-(3-{[7-propyl-3-(trifluoromethyl)-1,2-benzisoxazol-6-yl]oxy}propyl)urea,
(28) N′-(5-fluoro-2-pyridyl)-N-methyl-N-(3-{[7-propyl-3-(trifluoromethyl)-1,2-benzisoxazol-6-yl]oxy}propyl)urea;
(29) N′-(5-carbomethoxy-2-pyridyl)-N-methyl-N-(3-{[7-propyl-3-(trifluoromethyl)-1,2-benzisoxazol-6-yl]oxy}propyl)urea;
(30) N∝-(4-carboxy-2-pyridyl)-N-methyl-N-(3- ( [7-propyl-3-(trifluoromethyl)-1,2-benzisoxazol-6-yl]oxy}propyl)urea;
(31) N′-(1-oxo-2-pyridyl)-N-methyl-N-(3-{[7-propyl-3-(trifluoromethyl)-1,2-benzisoxazol-6-yl]oxy}propyl)urea;
(32) N′-(2-pyrimidinyl)-N-methyl-N-(3-{[7-propyl-3-(trifluoromethyl)-1,2-benzisoxazol-6-yl]oxy}propyl)urea;
(33) N′-(4-pyrimidinyl)-N-methyl-N-(3-{[7-propyl-3-(trifluoromethyl)-1,2-benzisoxazol-6-yl]oxy}propyl)urea;
(34) N′-(2-pyrazinyl)-N-methyl-N-(3-{[7-propyl-3-(trifluoromethyl)-1,2-benzisoxazol-6-yl]oxy}propyl)urea;
(35) N′-(5-tetrazolyl)-N-methyl-N-(3-{[7-propyl-3-(trifluoromethyl)-I,2-benzisoxazol-6-yl]oxy}propyl)urea;
   and the pharmaceutically acceptable salts, esters and tautomers thereof.

In a fourth embodiment are those compounds of Formula I wherein R⁵ is selected from oxazolyl and thiazoly, unsubstituted, mono- or polysubstituted with a substituent independently selected at each occurrence from the group consisting of —C₁₋₆alkyl and —CO₂R⁶. In a class of the fourth embodiment are those compounds of Formula I wherein R¹ is —CF₃; R² is —C₁₋₆ alkyl, and more particularly n-propyl; R³ is C₁₋₆alkyl, particularly selected from methyl and ethyl, and more particularly methyl; R⁴ is —H or methyl, and particularly —H; and Z is -n-propanediyl-. Examples of compounds within this embodiment and class include:
(36) N′-(5-methyl-3-oxazolyl)-N-methyl-N-(3-{[7-propyl-3-(trifluoromethyl)-1,2-benzisoxasol-6-yl]oxy}propyl)urea;
(37) N′-(2-thiazolyl)-N-methyl-N-(3-{[7-propyl-3-(trifluoromethyl)-1,2-benzisoxazol-6-yl]oxy}propyl)urea;
(38) N′-(3-methyl-5-oxazolyl)-N-methyl-N-(3-{[7-propyl-3-(trifluoromethyl)-1,2-benzisoxazol-6-yl]oxy}propyl)urea; and the pharmaceutically acceptable salts, esters and tautomers thereof.

In a fifth embodiment are those compounds of Formula I wherein R⁵ is phenyl, unsubstituted or monosubstituted with —CO₂R⁶. In a class of the fifth embodiment are those compounds of Formula I wherein R¹ is —CF₃; R² is —C₁₋₆ alkyl, and more particularly n-propyl; R³ is C₁₋₆alkyl, particularly selected from methyl and ethyl, and more particularly methyl; R⁴ is —H or methyl, and particularly —H; and Z is -n-propanediyl-. Examples of compounds within this embodiment and class include:
(39) N′-phenyl-N-methyl-N-(3-{[7-propyl-3-(trifluoromethyl)-1,2-benzisoxazol-6-yl]oxy}propyl)urea;
(40) N′-phenyl-N-ethyl-N-(3-{[7-propyl-3-(trifluoromethyl)-1,2-benzisoxazol-6-yl]oxy}propyl)urea;
(41) N′-(4-carbethoxyphenyl)-N-methyl-N-(3-{[7-propyl-3-(trifluoromethyl)-1,2-benzisoxazol-6-yl]oxy}propyl)urea;
(42) N′-(4-carboxyphenyl)-N-methyl-N-(3-{[7-propyl-3-(trifluoromethyl)-1,2-benzisoxazol-6-yl]oxy}propyl)urea; and the pharmaceutically acceptable salts, esters and tautomers thereof.

In a sixth embodiment are those compounds of Formula I wherein R⁵ is —H. In a class of the sixth embodiment are those compounds of Formula I wherein R¹ is —CF₃; R² is —C₁₋₆ alkyl, and more particularly n-propyl; R³ is C₁₋₆alkyl, particularly selected from methyl and ethyl, and more particularly methyl; R⁴ is —H or methyl, and particularly —H; and Z is -n-propanediyl-. An example of a compound within this embodiment and class includes:
(43) N-methyl-N-(3-{[7-propyl-3-(trifluoromethyl)-1,2-benzisoxazol-6-yl]oxy}propyl)urea, and the pharmaceutically acceptable salts, esters and tautomers thereof.

As used herein “alkyl” is intended to include both branched- and straight-chain saturated aliphatic univalent hydrocarbon groups having the specified number of carbon atoms. Examples of alkyl groups include, but are not limited to, methyl (Me), ethyl (Et), n-propyl (Pr), n-butyl (Bu), n-pentyl, n-hexyl, and the isomers thereof such as isopropyl (i-Pr), isobutyl (i-Bu), secbutyl (s-Bu), tertbutyl (t-Bu), isopentyl, isohexyl and the like. If there is no specified prefix with a named alkyl group, (such as “n-” for normal, ”s-” for sec, “t-” for tert, “i-” for iso) then it is intended that the alkyl goup is an n-alkyl group.

As used herein “alkanediyl” is intended to include both branched and straight-chain saturated aliphatic divalent hydrocarbon groups having the specified number of carbon atoms. Examples of alkanediyl groups include, but are not limited to, methanediyl, ethanediyl, propanediyl, butanediyl and the like. If there is no specified prefix (as described above) with a named alkanediyl group, then it is intended that the goup is an n-alkanediyl group.

As used herein “tetrazolyl” is

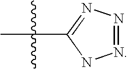

The term “oxazolyl” as used herein means: 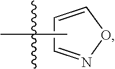
and is intended to include 3-oxazolyl, 4-oxazolyl, and 5-oxazolyl, optionally, mono- or disubstituted as defined in Formula I.

The term “thiazolyl” as used herein means: 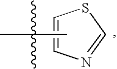
and is intended to include 2-thiazolyl, 4-thiazolyl, and 5-thiazolyl, optionally, mono or disubstituted as defined in Formula I.

The term “pyridyl” as used herein means: 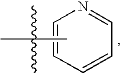
and is intended to include 2-pyridyl, 3-pyridyl, 4-pyridyl, 5-pyridyl, and 6-pyridyl, optionally, mono- or polysubstituted as defined in Formula I.

The term “pyrimidinyl” as used herein means: 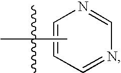
and is intended to include 2-pyrimidinyl, 4-pyrimidinyl, 5-pyrimidinyl, and 6-pyrimidinyl, optionally, mono-, or poly-substituted as defined in Formula I.

The term “pyrazinyl” as used herein means: 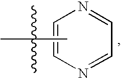
and is intended to include 2-pyrazinyl, 3-pyrazinyl, 5-pyrazinyl, and 6-pyrazinyl, optionally, mono-, or poly-substituted as defined in Formula I.

The terms “halo” and “halogen” are meant to include fluoro, chloro, bromo and iodo, unless otherwise noted. Fluoro and chloro are preferred.

Herein, the term “pharmaceutically acceptable salts” shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)aminomethane.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Examples of pharmaceutically acceptable esters include, but are not limited to, —C₁₋₄ alkyl and —C₁₋₄ alkyl substituted with phenyl-, dimethylamino-, and acetylamino. “C₁₋₄ alkyl” herein includes straight or branched aliphatic chains containing from 1 to 4 carbon atoms, for example methyl, ethyl, n-propyl, n-butyl, iso-propyl, sec-butyl and tert-butyl.

Under standard nomenclature used throughout this disclosure, the terminal portion of the designated side chain is described first, followed by the adjacent functionality toward the point of attachment. For example, a C1-5 alkylcarbonylamino C1-6 alkyl substituent is equivalent to

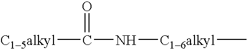

When referring to moieties which may optionally be substituted herein, e.g., alkyl groups, cycloalkyl groups, phenyl groups, heterocycloalkyl groups, and the like, the phrases used herein “unsubstituted, mono- or disubstituted with a substituent independently selected at each occurrence from the group consisting of and “unsubstituted, mono- or polysubstituted with a substituent independently selected at each occurrence from the group consisting of are intended to mean that the total number of substituents on the moiety overall may be zero, one or more than one, and that each carbon atom available for substitution in the given moiety may independently be unsubstituted or mono- or poly-substituted, with one or more substituents that are the same or different at each occurrence and which result in the creation of a stable structure. The term “poly-substituted” is intended to mean two or more substituents, e.g. di-, tri-, tetra-, penta-substitution and higher as appropriate, valence and stability permitting.

In choosing compounds of the present invention, one of ordinary skill in the art will recognize that the various substituents, i.e. R¹, R², etc., are to be chosen in conformity with well-known principles of chemical structure connectivity and stability. When any variable (e.g., R¹, R², etc.) occurs more than one time in any constituent or in formula I, its definition on each occurrence is independent of its definition at every other occurrence. Also, combinations of substituents and/or variables are permissible only if such combinations result in stable compounds. Compounds of Formula I may contain one or more asymmetric centers and can thus occur as racemates and racemic mixtures, single enantiomers, enantiomeric mixtures, diastereomeric mixtures and individual diastereomers. The present invention is meant to comprehend all such isomeric forms of the compounds of Formula I. All such isomeric forms of the compounds of Formula I are included within the scope of this invention. Some of the compounds described herein contain olefinic double bonds, and unless specified otherwise, are meant to include both E and Z geometric isomers.

The term “tautomers” embraces the standard meaning of the term, i.e. a type of isomerism in which two or more isomers are rapidly interconverted so that they ordinarily exist together in equilibrium. Tautomers include, e.g., compounds that undergo facile proton shifts from one atom of the compound to another atom of the compound. Some of the compounds described herein may exist as tautomers with different points of attachment of hydrogen. Such an example may be a ketone and its enol form known as keto-enol tautomers. The individual tautomers of the compounds of Formula I, as well as mixtures thereof, are included in the scope of this invention. By way of illustration, tautomers included in this definition include, but are not limited to:

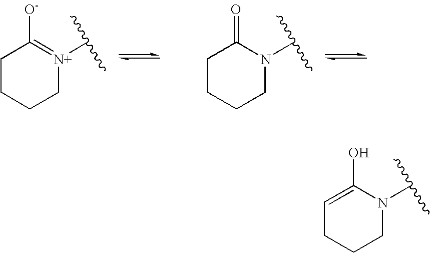

The term “rac” means racemic mixture, which is defined as a mixture comprised of equal amounts of enantiomers. If desired, racemic mixtures of compounds of Formula I may be separated by the coupling of a racemic mixture of the compounds of Formula I to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage and removal of the added chiral residue. The racemic mixture of the compounds of Formula I can also be separated directly by chromatographic methods utilizing chiral stationary phases, which methods are well known in the art. Alternatively, any enantiomer of a compound of the general Formula I may be obtained by stereoselective synthesis using optically pure starting materials or reagents of known configuration. Such methods are well known in the art.

Furthermore, some of the crystalline forms for compounds of the present invention may exist as polymorphs and as such are intended to be included in the present invention. In addition, some of the compounds of the instant invention may form solvates with water or common organic solvents. Such solvates are also encompassed within the scope of this invention.

Some abbreviations used herein are as follows: Ac is acetyl [CH3C(O)—]; PG is protecting group; LG is leaving group; Ac2O is acetic anhydride; 9-BBN is 9-borabicyclo[3.3.1]nonane; Pd(dba)2 is tris(dibenzylideneacetone)dipalladium; PdCl2dppf is dichlorobis-(triphenylphosphene) palladium; Ph is phenyl; PhMe is toluene; PPh3 is triphenylphosphine; Bn is benzyl; Me is methyl; Et is ethyl; EtOH is ethanol; EtOAc is ethyl acetate; Et3N is triethylamine; tBu is tert-butyl, PMB is para-methoxybenzyl; DMAP is 4-(dimethylamino)pyridine; DMF is N,N-dimethylformamide; DMSO is dimethyl sulfoxide; DIAD is diisopropylazodicarboxylate; Tf2O is triflic anhydride; Tf is triflate; TBAF is tetrabutyl ammonium fluoride; THF is tetrahydrofuran; TMS is trimethylsilyl; TBS is tert-butyldimethylsilyl; CDI is 1,1′-carbonyldiimidazole; HOBt is 1-hydroxybenzotriazole; EDAC (or EDC) is 1-ethyl-3-[3-(dimethylamino)propyl]-carbodiimide HCl; HCl is hydrochloric acid; Na DS is sodium hexamethyldisiliazide; LiHMDS is lithium hexamethyldisiliazide; DIBAL is diisobutylaluminum hydride; TPAP is tetrapropylammonium perruthenate; NMO is N-methylmorpholine N oxide; MsCl is methanesulfonyl chloride; TFA is trifluoroacetic acid; HPLC is high performance liquid chromatography; NaOAc is sodium acetate; NaOtBu is sodium tert-butoxide; TLC is thin layer chromatography; RT is room temperature; N is normal; mmol is millimole; and M is molar.

The compounds of this invention can be prepared employing the following general procedures. Benzisoxazole intermediates may be prepared from commercially available or readily accessible resorcinols as shown in Scheme 1 or alternate synthetic pathways as reported in the literature. See for example: Shutske, G. M. et al., J. Med. Chem., 25 (1), 36 (1982); Poissonnet, G. Synth. Commun., 27 (22), 3839-3846 (1997); Crabbe, P. et al., J. Chem. Soc., Perkin Trans 1, 1973, 2220.

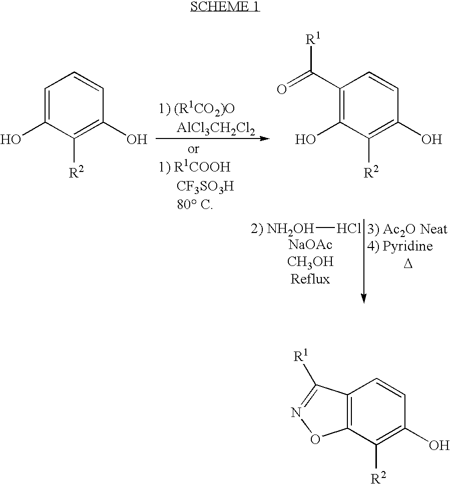

For maximum flexibility these phenolic benzisoxazoles may be converted to intermediate amine reagents for condensation with a variety of acylating agents, as shown in Scheme 2 below. Formation of the secondary amine typically occurs in the presence of a large excess of the amine partner. Acylation with completely elaborated acylating agents, such as isocyanates or carbamoyl chlorides, can lead directly to the desired compounds. Some acylating agents may contain other protected functionalities, such as an ester which will need to be de-protected in a final step. Synthesis of those isocyanates or carbamoyl reagents which are not commercially available can be readily synthesized by common procedures known to those skilled in the art. In some cases where R₅ is not equal to hydrogen, alkylation of the urea on the un-substituted nitrogen is readily accomplished with a base such as NaH and an alkylating agent such as an alkyl halide.

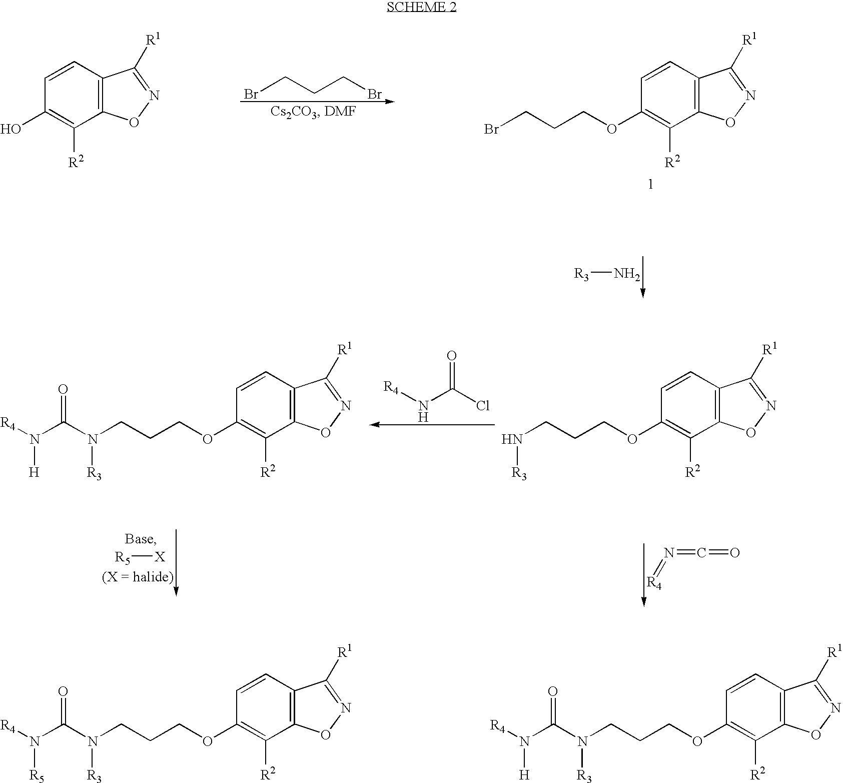

As an alternative approach, an alternate order of addition may be used to construct these compounds as indicated in Scheme 3 below.

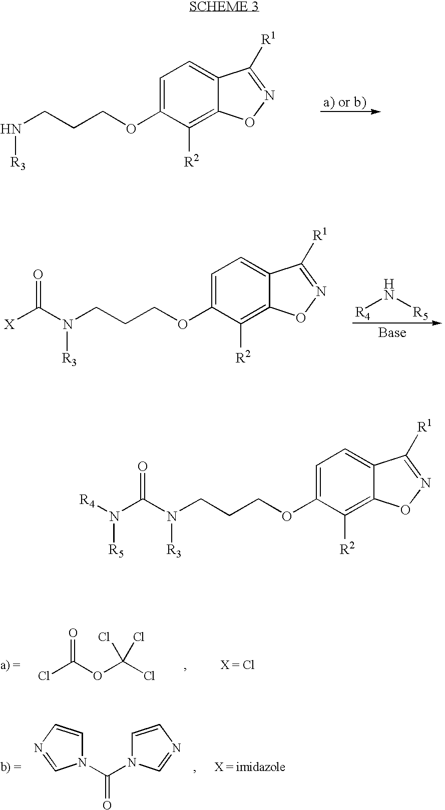

The intermediate amine fragment is converted to a carbamoyl chloride or carbamoylimidazolide using diphosgene or 1,1′-carbonyldiimidazole as shown. In the schemes above, R², R³ and R⁴ are alkyl, aryl or heteroaryl substituents as desired. 10 The instant invention provides methods for treating lipid disorders, particularly for treating below-desired plasma HDL cholesterol levels, as well as for treating and/or reducing the risk for diseases and conditions affected by LXR activity, comprising administering a therapeutically effective amount of a compound of Formula I to a person in need of such treatment. Any patient having a depressed plasma HDL cholesterol level, or desiring to increase their HDL cholesterol level may use this treatment. Particularly suitable patients in need of such treatment are those whose plasma HDL cholesterol level is depressed, i.e., below the clinically desirable level. Currently, the clinically desirable HDL cholesterol level is considered to be a minimum of 40 mg/dl in men and about 50 mg/dl or higher in women. NCEP guidelines define 60 mg/dl as a desirable, cardioprotective, HDL level.

The method of this invention also serves to prevent lipid accumulation in, or remove lipids from, tissue deposits such as atherosclerotic plaques or xanthomas in a patient with atherosclerotic disease manifest by clinical signs such as angina, claudication, bruits, one that has suffered a myocardial infarction or transient ischemic attack, or one diagnosed by angiography, sonography or MRI.

Further provided are methods for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a mammal, including a human, who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

A compound of Formula I may be administered to prevent or reduce the risk of occurrence, or recurrence where the potential exists, of a coronary heart disease event, a cerebrovascular event, and/or intermittent claudication. Coronary heart disease (CHD) events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of Formula I to a patient at risk for such an event. The patient may or may not have atherosclerotic disease at the time of administration, or may be at risk for developing it.

Persons to be treated with the instant therapy include those with dyslipidemic conditions including depressed or below-desirable plasma levels of HDL cholesterol, as well as those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein cholesterol, and a family history of atherosclerotic cardiovascular disease. Published guidelines for determining those who are at risk of developing atherosclerotic disease can be found in: Executive Summary of the Third Report of the National Cholesterol Education Program (NCEP) Expert Panel on Detection, Evaluation, and Treatment of High Blood Cholesterol in Adults (Adult Treatment Panel III), JAMA, 2001; 285 pp. 2486-2497. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by reverse cholesterol transport.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical agent that will prevent or reduce the risk of occurrence of the biological or medical event that is sought to be prevented in a tissue, a system, animal or human by a researcher, veterinarian, medical doctor or other clinician. Particularly, the dosage amount of a compound of Formula I that a patient receives can be selected so as to achieve the amount of lipid level modification desired, particularly to achieve a desired level of HDL cholesterol. The dosage a patient receives may also be titrated over time in order to reach a target lipid profile. The dosage regimen utilizing a compound of Formula I is selected in accordance with a variety of factors including type, species, age, weight, sex and medical condition of the patient; the severity of the condition to be treated; the potency of the compound chosen to be administered; drug combinations; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition.

An effective amount of compound for use in the method of this invention is about 0.01 mg/kg to about 30 mg/kg of body weight per day, or about 0.7 mg to about 2100 mg per patient in single or divided doses per day. More particularly, an amount of about 7 mg to about 1050 mg per patient in single or divided doses per day can be administered. However, dosage amounts will vary depending on factors as noted above, including the potency of the particular compound. Although the active drug of the present invention may be administered in divided doses, for example from one to four times daily, a single daily dose of the active drug is preferred.

Administration of the active drug can be via any pharmaceutically acceptable route and in any pharmaceutically acceptable dosage form. This includes the use of oral conventional rapid-release, time controlled-release and delayed-release (such as enteric coated) pharmaceutical dosage forms. The active drug employed in the instant therapy can be administered in such oral forms as tablets, capsules, pills, powders, granules, elixirs, tinctures, suspensions, syrups, and emulsions. Additional suitable pharmaceutical compositions for use with the present invention are known to those of ordinary skill in the pharmaceutical arts; for example, see RemingtoN's Pharmaceutical Sciences, Mack Publishing Co., Easton, Pa. Oral formulations are preferred.

In the methods of the present invention, the active drug is typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants, for example butylated hydroxyanisole (BHA), 2,6-di-tert-butyl-4-methylphenol (BHT), propyl gallate, sodium ascorbate, citric acid, calcium metabisulphite, hydroquinone, and 7-hydroxycoumarin, can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drug can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active drug may also be delivered by the use of monoclonal antibodies as individual carriers to which the compound molecules are coupled. The active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, the active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

In a broad embodiment, any suitable additional active agent or agents may be used in combination with the compound of Formula I in a single dosage formulation, or may be administered to the patient in a separate dosage formulation, which allows for concurrent or sequential administration of the active agents. One or more additional active agents may be administered with a compound of Formula I. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin, particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as ZD-4522, (CRESTOR®; see U.S. Pat. No. 5,260,440, and Drugs of the Future, 1999, 24(5), pp. 511-513); HMG-CoA synthase inhibitors; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; cholesteryl ester transfer protein (CETP) inhibitors, such as Pfizer's CP 529,414 (WO/0038722 and EP 818448) and Pharmacia's SC-744 and SC-795; niacin; probucol; bile acid sequestrants; LDL (low density lipoprotein) receptor inducers; platelet aggregation inhibitors, for example glycoprotein IIb/IIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate, other fibrate class PPARα agonists and gemfibrozil; PPAR dual α/γ agonists including aryloxyacetic acids (see U.S. Pat. No. 6,569,879), 2-aryloxy-2-arylalkanoic acids (see WO02/064094) and benzopyrancarboxylic acids (see U.S. Patent Publication No. 20020103242); vitamin B6 (also known as pyridoxine) and the pharmaceutically acceptable salts thereof such as the HCl salt; vitamin B12 (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABCA1 gene expression; FXR ligands including both inhibitors and agonists; bisphosphonate compounds such as alendronate sodium; and cyclooxygenase-2 inhibitors such as rofecoxib and celecoxib.

Still another type of agent that can be used in combination with the compounds of this invention are cholesterol absorption inhibitors. Cholesterol absorption inhibitors block the movement of cholesterol from the intestinal lumen into enterocytes of the small intestinal wall. This blockade is their primary mode of action in reducing serum cholesterol levels. These compounds are distinct from compounds which reduce serum cholesterol levels primarily by mechanisms of action such as acyl coenzyme A-cholesterol acyl transferase (ACAT) inhibition, inhibition of triglyceride synthesis, MTP inhibition, bile acid sequestration, and transcription modulation such as agonists or antagonists of nuclear hormones. Cholesterol absorption inhibitors are described in U.S. Pat. No. 5,846,966, U.S. Pat. No. 5,631,365, U.S. Pat. No. 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, WO 02/066464, WO 00/63703, WO /0060107, WO 00/38725, WO 00/34240, WO 00/20623, WO 97/45406, WO 97/16424, WO 97/16455, and WO 95/08532, the entire contents of all of which are hereby incorporated by reference.

An exemplary cholesterol absorption inhibitor is ezetimibe, sold in the U.S. under the tradename ZETIA®, which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966 and shown below as

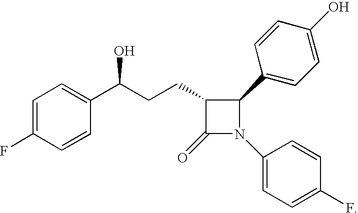

Additional exemplary hydroxy-substituted azetidinone cholesterol absorption inhibitors are specifically described in U.S. Pat. No. 5,767,115, column 39, lines 54-61 and column 40, lines 1-51 (hereby incorporated by reference), represented by the formula 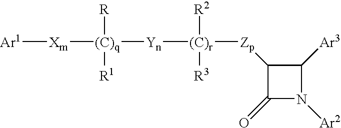
as defined in column 2, lines 20-63 (hereby incorporated by reference).

Additional exemplary C-glycosidic azetidinone cholesterol absorption inhibitors are disclosed in WO 02/066464 (hereby incorporated by reference in its entirety), represented by the formula 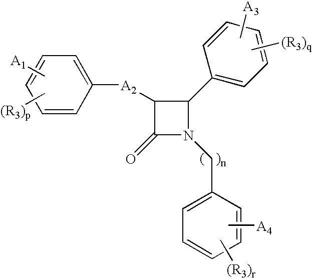
as defined on pages 3 line 24- page 5 line 3. These and other cholesterol absorption inhibitors can be identified according to the assay of hypolipidemic compounds using the hyperlipidemic hamster described in U.S. Pat. No. 5,767,115, column 19, lines 47-65 (hereby incorporated by reference), in which hamsters are fed a controlled cholesterol diet and dosed with test compounds for seven days. Plasma lipid analysis is conducted and data is reported as percent reduction of lipid versus control.

Therapeutically effective amounts of cholesterol absorption inhibitors include dosages of from about 0.01 mg/kg to about 30 mg/kg of body weight per day, preferably about 0.1 mg/kg to about 15 mg/kg. For an average body weight of 70 kg, the dosage level is therefore from about 0.7 mg to about 2100 mg of drug per day, e.g. 10, 20, 40, 100 or 200 mg per day, preferably given as a single daily dose or in divided doses two to six times a day, or in sustained release form. This dosage regimen may be adjusted to provide the optimal therapeutic response when the cholesterol absorption inhibitor is used in combination with a compound of the instant invention.

According to a further aspect of the present invention there is provided the use of a compound of Formula I for the manufacture of a medicament for the treatment, prevention, or reduction in risk of developing a LXR receptor mediated disease. A therapeutically or prophylactically effective amount, as appropriate, of a compound of Formula I can be used for the preparation of a medicament useful for treating lipid disorders, particularly for treating depressed HDL cholesterol levels as well as for treating and/or reducing the risk for diseases and conditions affected by agonism of LXR and affected by reverse cholesterol transport, preventing or reducing the risk of developing atherosclerotic disease, halting or slowing the progression of atherosclerotic disease once it has become clinically manifest, and preventing or reducing the risk of a first or subsequent occurrence of an atherosclerotic disease event. For example, the medicament may be comprised of about 0.7 mg to about 2100 mg of a compound of Formula I, or more particularly about 7 mg to about 1050 mg. The medicament comprised of a compound of Formula I may also be prepared with one or more additional active agents, such as those described supra.

As used herein, the term LXR includes all subtypes of this receptor, e.g., designated as LXRα (NR1H3) and LXRβ (NR1H2, Cell 97, 161-163, 1999). The compounds of Formula I are LXR ligands and individually may vary in their selectivity for one or the other of LXRα and LXRβ, or they may have mixed binding affinity for both LXRα and LXRβ. More particularly, the tested compounds included within the scope of this invention have an IC₅₀ less than or equal to 2 μM for at least one of either the LXRα or LXRβ receptors employing the LXR radioligand competition scintillation proximity assays described below in the Example section. Preferred tested compounds of Formula I bind to the human LXRα receptor and have an IC₅₀ less than or equal to 300 nM for the LXRα receptor.

Compound A is used in the following assays and has the following structural formula: 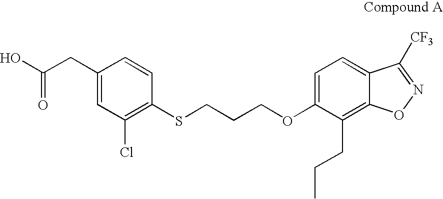
Compound A and related compounds are disclosed along with methods for making them in WO97/28137 herein incorporated by reference in its entirety (U.S. Ser. No. 08/791211, filed Jan. 31, 1997).

The compounds in the following examples were characterized using ¹H NMR at 400 or 500 MHz field strength, and/or by ESI mass spectroscopy (MS).

### EXAMPLE 1

Radioligand Competition Binding Scintillation Proximity Assays

Preparation of Recombinant Human LXRα and LXRβ:

Human LXRα and LXRβ were expressed as GST-fusion proteins in E. coli. The ligand binding domain cDNAs for human LXRα (amino acids 164-447) and human LXRβ (amino acids 149-455) were subcloned into the pGEX-KT expression vector (Pharmacia). E. coli containing the respective plasmids were propagated, induced, and harvested by centrifugation. The re-suspended pellet was broken in a French press and debris was removed by centrifugation. Recombinant human LXR receptors were purified by affinity chromatography on glutathione sepharose and receptor was eluted with glutathione. Glycerol was added to a final concentration of 50% to stabilize the receptor and aliquots were stored at −80° C.

Binding to LXRα: For each assay, an aliquot of human GST-LXRα receptor was incubated in a final volume of 100 μl SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 μg/ml anti-GST antibody (Amersham Pharmacia Biotech, Inc.), 0.1% non-fat dry milk and 25 nM [³H₂]Compound A (13.4 Ci/mmole), ±test compound. After incubation for ˜16 h at 15° C. with shaking, the assay plates were counted in a Packard Topcount. In this assay the K_{d} for Compound A for LXRα is ≈15 nM.

Binding to LXRβ: For each assay, an aliquot of human GST-LXRβ ligand binding domain receptor was incubated in a final volume of 100 μl SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 μg/ml anti-GST antibody (Amersham Pharmacia Biotech, Inc.) 0.1% non-fat dry milk and 25 nM [³H₂]Compound A (13.4 Ci/mmole), ±test compound. After incubation for ˜16 h at 15° C. with shaking, the assay plates were counted in a Packard Topcount. In this assay the K_{d} for Compound A for LXRβ is ˜10 nM.

Results: Representative tested compounds of Formula I are ligands for human LXRα and/or human LXRβ, each having an IC₅₀≦1,800 nM for at least one of the LXRα receptor or the LXRβ receptor, and preferred tested compounds having an IC₅₀ of 300 nM or less for at least one of the LXRα receptor or the LXRβ receptor.

### EXAMPLE 2

Transactivation Assay

Plasmids: Expression constructs were prepared by inserting the ligand binding domain (LBD) of human LXRα and LXRβ cDNAs adjacent to the yeast GAL4 transcription factor DNA binding domain (DBD) in the mammalian expression vector pcDNA3 to create pcDNA3-LXRα/GAL4 and pcDNA3-LXRβ/GAL4, respectively. The GAL4-responsive reporter construct, pUAS(5X)-tk-luc, contained 5 copies of the GAL4 response element placed adjacent to the thymidine kinase minimal promoter and the luciferase reporter gene. The transfection control vector, pEGFP-N1, contained the Green Fluorescence Protein (GFP) gene under the regulation of the cytomegalovirus promoter.

Assay: HEK-293 cells were seeded at 40,000 cells/well in 96 well plates in Dulbecco's modified Eagle medium (high glucose) containing 10% charcoal stripped fetal calf serum, 100 units/ml Penicillin G and 100 μg/ml Streptomycin sulfate at 37° C. in a humidified atmosphere of 5% CO₂. After 24 h, transfections were performed with Lipofectamine (Gibco-BRL, Gaithersburg, Md.) according to the instructions of the manufacturer. In general, transfection mixes contained 0.002 μg of LXRα/GAL4 or LXRβ/GAL4 chimeric expression vectors, 0.02 μg of reporter vector pUAS(5X)-tk-luc and 0.034 μg of pEGFP-N1 vector as an internal control of transfection efficiency. Compounds were characterized by incubation with transfected cells for 48 h across a range of concentrations. Cell lysates were prepared from washed cells using Cell Lysis Buffer (Promega) according to the manufacturer's directions. Luciferase activity in cell extracts was determined using Luciferase Assay Buffer (Promega) in a ML3000 luminometer (Dynatech Laboratories). GFP expression was determined using the Tecan Spectrofluor Plus at excitation wavelength of 485 nm and emission at 535 nm. Luciferase activity was normalized to GFP expression to account for any variation in efficiency of transfection.

Results: Representative tested compounds of Formula I for LXRα transactivation had an EC₅₀ of ≦5,500 nM for at least one of the LXRα receptor or the LXRβ receptor, and preferred tested compounds had an EC₅₀ of 1,000 nM or less for at least one of the LXRα receptor or the LXRβ receptor.

### EXAMPLE 3

To assess the relevant biological activity of the LXR agonists, certain compounds were tested for their ability to increase cholesterol efflux from cultured human cells, as described by Sparrow et al., JBC, 277, 10021-10027, Mar. 22, 2002. Caco-2 cells, which are of human origin, were obtained from ATCC and grown in Opti-MEM (Gibco #51985-034) containing 10% FCS, non-essential amino acids (Gibco #11140-050), and vitamins (Gibco # 11120-052). Caco-2 cells were plated at 100,000 cells/well in 48-well plates. After four days the cells had reached confluence, and were then labeled with ³H-cholesterol by incubation for 24 hours in fresh growth media containing ³H-cholesterol (10 μCi/mi). Following labeling with ³H-cholesterol, cells were washed and incubated an additional 24 hours in serum-free media containing 1 mg/ml BSA, to allow for equilibration of ³H-cholesterol with intracellular cholesterol. Cholesterol efflux was initiated by adding 10 μg/ml apoA-I, with or without compound, in serum-free medium. Compounds were added to cell culture medium from DMSO solutions, and control cells received an equivalent amount of DMSO, never exceeding 0.1%. After 24 hours, media were harvested and cells dissolved in 0.1 M NaOH. Media were briefly centrifuged to remove non-adherent cells, and then aliquots of both the supernatants and the dissolved cells were subjected to liquid scintillation spectrometry to determine radioactivity. Cholesterol efflux is expressed as a percentage, calculated as (³H-cholesterol in riedium/(³H-cholesterol in medium+³H-cholesterol in cells))×100.

Table 1 shows cholesterol efflux results for compound (18), N′-(2-pyridyl)-N-methyl-N-(3-{[7-propyl-3-(trifluoromethyl)-1,2-benzisoxazol-6-yl]oxy}propyl)urea (made in Example 25). Results are given as mean of quadruplicate incubations.

TABLE 1

| Compound (18) | |
|---|---|
| concentration (nM) | % cholesterol efflux |
| 0 | 4.3 |
| 30 | 3.4 |
| 100 | 3.5 |
| 300 | 4.9 |
| 1000 | 5.7 |
| 3000 | 6.8 |
| 10000 | 5.6 |

Table 2 show cholesterol efflux results for compound (26), N′-(4-carboxy-2-pyridyl)-N-methyl-N-(3-{[7-propyl-3-(trifluoromethyl)-1,2-benzisoxazol-6-yl]oxy}propyl)urea (made in Example 43). Results are given as mean of quadruplicate incubations.

TABLE 2

| Compound (26) | |
|---|---|
| concentration (nM) | % cholesterol efflux |
| 0 | 3.9 |
| 30 | 3.1 |
| 100 | 3.3 |
| 300 | 3.7 |
| 1000 | 4.6 |
| 3000 | 5.7 |
| 10000 | 6.6 |

### EXAMPLE 4

Preparation of 6-hydroxy-7-propyl-3-(trifluoromethyl)-1,2-benzisoxazole

Step 1: Preparation of 2,4-dihydroxy-3-propyl-1′,1′,1′-trifluoroacetophenone.

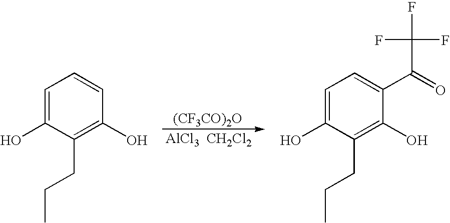

A solution of 2-porpylresorcinol (5.0 grams) and trifluoroacetic anhydride (9.6 mL) in 1,2-dichloroethane (30.0 mL) was treated with aluminum chloride (4.38 grams). This mixture was stirred overnight. The reaction mixture was partitioned between methylene chloride and water. The organic phase was dried over sodium sulfate and filtered. The solvent was evaporated and the resulting solid was recrystallized from methylene chloride and cyclohexane (1:1) to give the title compound.

Selected Signals: ¹H NMR (CDCl₃) δ 7.59 (d, 1H), 6.24 (d, 1H), 5.92 (s, 1H), 2.63 (t, 2H), 1.74 (s, 1H), 1.58 (m, 2H), 0.98 (t, 3H).
Step 2: Preparation of 6-hydroxy-7-propyl-3-(trifluoromethyl)-1,2-benzisoxazole

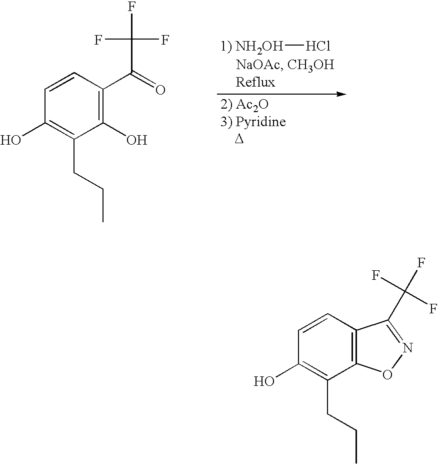

A mixture of 2,4-dihydroxy-3-propyl-1′,1′,1′-trifluoroacetophenone (2.5 grams), sodium acetate (4.18 grams), hydroxylamine hydrochloride (3.59 grams) and methanol (80 mL) was refluxed overnight. The solvent was then evaporated and the resulting solid was partitioned between ethyl acetate and pH 7 buffer. The organic phase was separated and washed with brine. The organic phase was dried over sodium sulfate and the solvent was evaporated to give an oil. The oil was then dissolved in acetic anhydride. The solution was stirred for two hours, then the acetic anhydride was evaporated in vacuo. The residue was partitioned between ethyl acetate and pH 7 buffer and the organic phase was dried over sodium sulfate. The organic phase was evaporated to give an oil. The oil was dissolved in pyridine and refluxed overnight. The solvent was evaporated in vacuo to give an oil which was chromatographed on silica gel using ethyl acetate and hexane (1:4) to give the title compound.

Selected Signals: ¹H NMR (CDCl₃) δ 7.46 (d, 1H), 6.92 (d, 1H), 5.42 (bs, 1H), 2.89 (t, 2H), 1.74 (m, 2H), 0.98 (t, 3H).

### EXAMPLE 5

Preparation of 7-propyl-3-(trifluoromethyl)-6-(3-bromopropyloxy)-1,2-benzisoxazole.

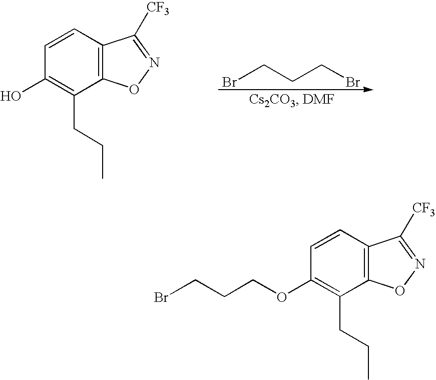

To a DMF solution (50 mL) of 6-hydroxy-7-propyl-3-(trifluoromethyl)-1,2-benzisoxazole as prepared in Example 4 (5 grams, 20.4 mmol) was added 1,3-dibromopropane (10 mL, 98.5 mmol), followed by cesium carbonate (10 grams, 30.7 mmol). The mixture was stirred at room temperature overnight. After aqueous/ether work-up and purification by chromatography on silica gel (hexanes: 2.5% ethyl acetate), the titled compound was obtained.

Selected Signals: ¹H NMR (CDCl₃); δ 7.59 (d, 2H, J=8.8 Hz), 7.10 (d, 2H, J=8.8 Hz), 4.27 (t, 2H, J=5.8 Hz), 3.66 (t, 2H, J=6.4 Hz), 2.93 (t, 2H, J=7.5 Hz), 2.41 (pent, 2 H, J=6.0 Hz), 1.72 (sext, 2H, J=7.5 Hz), 0.99 (t, 3H, J=7.5 Hz).

### EXAMPLE 6

Preparation of 7-propyl-3-(trifluoromethyl)-6-( 3-methylaminopropyloxy)-1,2-benzisoxazole.

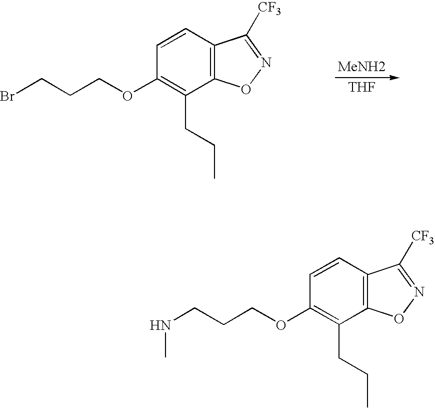

To a THF solution (50 mL) of 7-propyl-3-(trifluoromethyl)-6-(3-bromopropyloxy)-1,2-benzisoxazole as prepared in Example 5 (0.5 grams, 1.35 mmol) was added methylamine (7 mL, ˜2 M THF, ˜14 mmol) and the mixture was stirred at room temperature overnight. After aqueous/ether work-up and purification by chromatography on silica gel methanol: methylene chloride (10/90) 2% NH₄OH, the titled compound was obtained.

Selected Signals: ¹H NMR (CDCl₃); δ 7.59 (d, 2H, J=8.8 Hz), 7.10 (d, 2H, J=8.8 Hz), 4.27 (t, 2H, J=5.8 Hz), 3.66 (t, 2H, J=6.4Hz), 2.93 (t, 2H, J=7.5 Hz), 2.41 (pent, 2 H, J=6.0 Hz), 1.72 (sext, 2H, J=7.5 Hz), 0.99 (t, 3H, J=7.5 Hz).

### EXAMPLE 7

Preparation of 7-propyl-3-(trifluoromethyl)-6-(3-ethylaminopropyloxy)-1,2-benzisoxazole

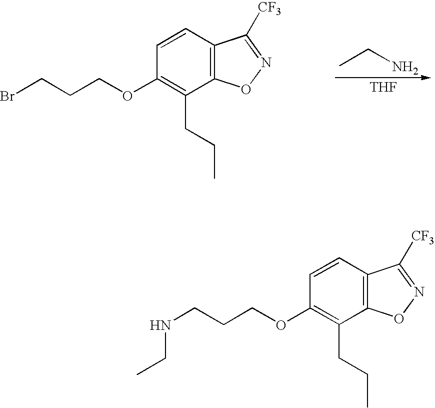

To a THF solution (50 mL) of 7-propyl-3-(trifluoromethyl)-6-(3-bromopropyloxy)-1,2-benzisoxazole as prepared in Example 5 (0.32 grams, 0.87 mmol) was added ethylamine (2M THF, 4.4 mL, 8.7 mmol) and the mixture was stirred at room temperature overnight. After aqueous/ether work-up and purification by chromatography on silica gel methanol: methylene chloride (10:90), 2% NH₄OH aq, the titled compound was obtained.

Selected Signals: ¹H NMR (CDCl₃); δ 7.59 (d, 2H, J=8.8 Hz), 7.10 (d, 2H, J=8.8 Hz), 4.27 (t, 2H, J=5.8 Hz), 3.66 (t, 2H, J=6.4 Hz), 2.93 (t, 2H, J=7.5 Hz), 2.41 (pent, 2 H, J=6.0 Hz), 1.72 (sext, 2H, J=7.5 Hz), 0.99 (t, 3H, J=7.5 Hz).

MS: m/z=331 (M+H).

### EXAMPLE 8

Preparation of N,N′-dimethyl-N-(3-{[7-propyl-3-(trifluoromethyl)-1,2-benzisoxazol-6-yl]oxy}propyl)urea

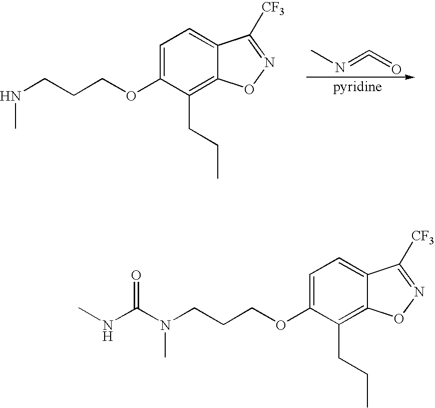

To a solution of 7-propyl-3-(trifluoromethyl)-6-(3-methylaminopropyloxy)-1,2-benzisoxazole prepared according to the procedure of Example 6 (22.5 mg, 0.07 mmol) in pyridine (2 mL) was added methyl isocyanate (4.75 μL, 0.08 mmol). The reaction mixture was stirred at room temperature overnight. The solvent was evaporated in vacuo, and the residue was purified by chromatography on silica gel using methanol: methylene chloride (1:19) to give the title compound.

¹H NMR (CDCl₃) δ 7.55 (d, 1H, J=8.5 Hz), 7.05 (d, 1H, J=9.0), 4.44 (br, 1H), 4.13 (t, 2H, J=6.0), 3.48 (t, 2H, J=7.0), 2.91 (m, 5H), 2.77 (m, 3H), 2.08 (m, 2H), 1.70 (m, 2H), 0.94 (m, 3H).

MS: m/z=374 (M+H).

### EXAMPLE 9

Preparation of N′-ethyl-N-methyl-N-(3-{[7-propyl-3-(trifluoromethyl)-1,2-benzisoxazol-6-yl]oxy}propyl)urea.

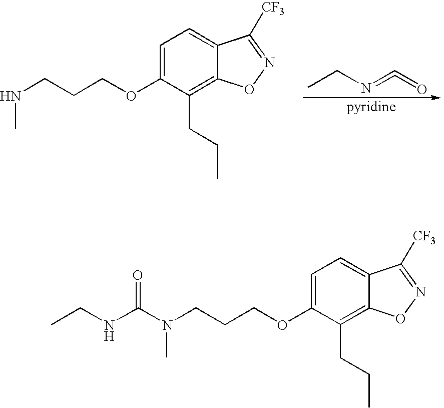

To a solution of 7-propyl-3-(trifluoromethyl)-6-(3-bromopropyloxy3-methylaminopropyloxy)-1,2-benzisoxazole (40.5 mg, 0.128 mmol) in pyridine (2 mL) was added ethyl isocyanate (11.4 μL, 0.14 mmol). The reaction mixture was stirred at room temperature overnight. The solvent was evaporated in vacuo, and the residue was purified by chromatography on silica gel using methanol: methylene chloride (5:95), 1% NH₄OH aq, to give the title compound.

¹H NMR (CDCl₃) δ 7.53 (d, 1H, J=9.0 Hz), 7.05 (d, 1H, J=8.5), 4.45 (br, 1H), 4.12 (m, 2H), 3.48 (t, 2H, J=7.0), 3.21 (m, 2H), 2.90 (m, 5H), 2.07 (m, 2H), 1.70 (m, 2H), 1.04 (t, 3H, J=7.0), 0.95 (m, 3H).

MS: m/z=388 (M+H).

### EXAMPLE 10

Preparation of N′-propyl-N-methyl-N-(3-{[7-propyl-3-(trifluoromethyl)-1,2-benzisoxazol-6-yl]oxy}propyl)urea.

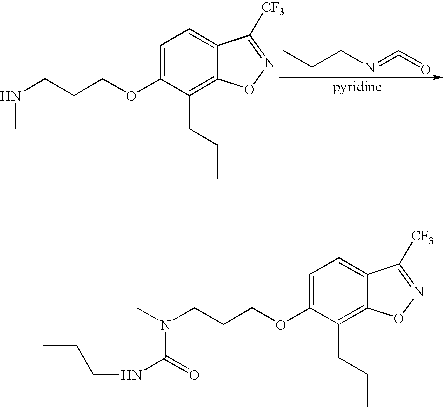

To a solution of 7-propyl-3-(trifluoromethyl)-6-(3-bromopropyloxy3-methylaminopropyloxy)-1,2-benzisoxazole (40.1 mg, 0.127 mmol) in pyridine (2 mL) was added propyl isocyanate (13.3 μL, 0.14 mmol). The reaction mixture was stirred at room temperature overnight. The solvent was evaporated in vacuo, and the residue was purified by chromatography on silica gel using methanol: methylene chloride (5:95) to give the title compound.

¹H NMR (CDCl₃) δ 7.52 (d, 1H, J=9.0 Hz), 7.04 (d, 1H, J=8.5), 4.50 (br, 1H), 4.11 (m, 2H), 3.48 (t, 2H, J=7.0), 3.12 (m, 2H), 2.90 (m, 5H), 2.07 (m, 2H), 1.70 (m, 2H), 0.95 (t, 3H, J=6.0), 0.85 (m, 3H).

MS: m/z=402 (M+H).

### EXAMPLE 11

Preparation of N′-isopropyl-N-methyl-N-(3-{[7-propyl-3-(trifluoromethyl)-1,2-benzisoxazol-6-yl]oxy}propyl)urea.

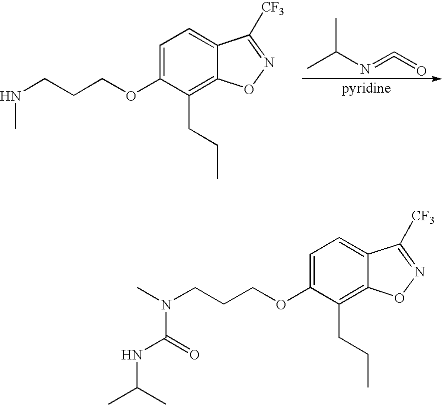

To a solution of 7-propyl-3-(trifluoromethyl)-6-(3-bromopropyloxy3-methylaminopropyloxy)-1,2-benzisoxazole (33.0 mg, 0.104 mmol) in pyridine (2 mL) was added isopropyl isocyanate (11.5 μL, 0.115 mmol). The reaction mixture was stirred at room temperature overnight. The solvent evaporated in vacuo, and the residue was purified by chromatography on silica gel using methanol : methylene chloride (2:98) to give the title compound.

¹H NMR (CDCl₃) δ 7.53 (d, 1H, J=8.5 Hz), 7.05 (d, 1H, J=9.0), 4.22 (m, 1H), 4.12 (t, 2H, J=6.0), 3.93 (m, 1H), 3.48 (t, 2H, J=6.5), 3.21 (m, 2H), 2.90 (m, 5H), 2.07 (t, 2H, J=6.0), 1.70 (m, 2H), 1.06 (d, 6H, J=6.0), 0.95 (t, 3H, J=6.0).

MS: m/z=402 (M+H).

### EXAMPLE 12

Preparation of N′-phenyl-N-methyl-N-(3-{[7-propyl-3-(trifluoromethyl)-1,2-benzisoxazol-6-yl]oxy}propyl)urea.

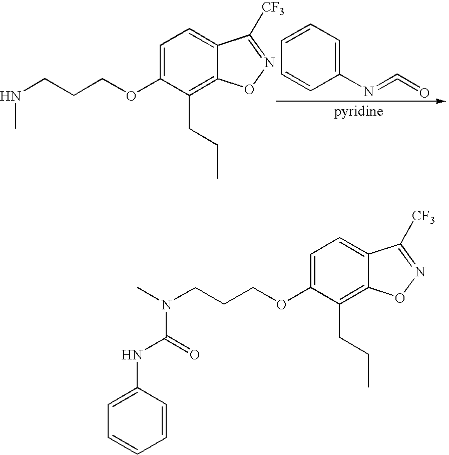

To a solution of 7-propyl-3-(trifluoromethyl)-6-(3-bromopropyloxy3-methylaminopropyloxy)-1,2-benzisoxazole (41.9 mg, 0.133 mmol) in pyridine (2 mL) was added phenyl isocyanate (16.2 μL, 0.146 mmol). The reaction mixture was stirred at room temperature overnight. The solvent was evaporated in vacuo, and the residue was purified by chromatography on silica gel using ethyl acetate: hexanes (1:1) to give the title compound.

¹H NMR (CDCl ₃) δ 7.54 (d, 1H, J=8.5 Hz), 7.28-6.97 (m, 5 H), 7.06 (d, 1H, J=9.0), 6.43 (br, 1H), 4.18 (t, 2H, J=5.0), 3.61 (t, 2H, J=7.0), 3.06 (s, 3H), 2.93 (t, 2H, J=7.5), 2.17 (m, 2H), 1.72 (m, 2H), 0.96 (t, 3H, J=7.5).

MS: m/z=436 (M+H).

### EXAMPLE 13

Preparation of N′-(2-carbethoxyethyl)-N-methyl-N-(3-{[7-propyl-3-(trifluoromethyl)-1,2-benzisoxazol-6-yl]oxy}propyl)urea and N′-(2-carboxyethyl)-N-methyl-N-(3-{[7-propyl-3-(trifluoromethyl)-1,2-benzisoxazol-6-yl]oxy}propyl)urea.

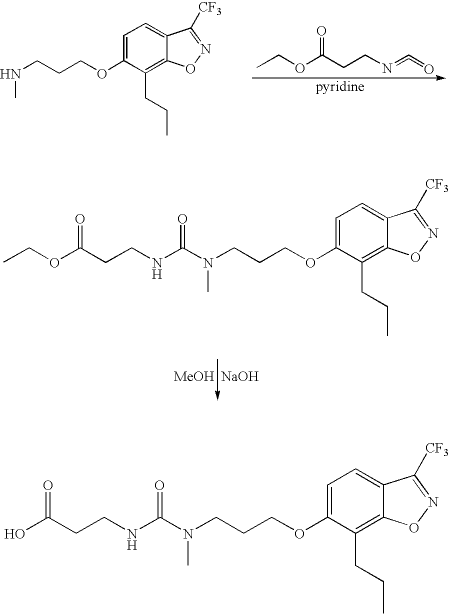

To a solution of 7-propyl-3-(trifluoromethyl)-6-(3-bromopropyloxy3-methylaminopropyloxy)-1,2-benzisoxazole (33.6 mg, 0.12 mmol) in pyridine (2 mL) was added ethyl 3-isocyanatopropionate (17.4 μL, 0.13 mmol). The reaction mixture was stirred at room temperature overnight. The solvent was evaporated in vacuo, and the residue was purified by chromatography on silica gel using methanol and methylene chloride (1:49) to give the urea ester N′-(2-carbethoxyethyl)-N-methyl-N-(3-{[7-propyl-3-(trifluoromethyl)-1,2-benzisoxazol-6-yl]oxy}propyl)urea. The urea ester (30.1 mg, 0.07 mmol) was dissolved in MeOH (1 mL) and NaOH aq (1 N, 1 mL) was added in to the solution and stirred at room temperature overnight. The reaction mixture was neutralized with 1N HCl and purified by prep-RP8 HPLC (0.1% TFA in acetonitrile (10-100% gradient)/H₂O) to afford N′-(2-carboxyethyl)-N-methyl-N-(3-{[7-propyl-3-(trifluoromethyl)-1,2-benzisoxazol-6-yl]oxy}propyl)urea.

¹H NMR (CDCl₃) δ 8.82(br, 1H), 7.54 (d, 1H, J=9.0 Hz), 7.04 (d, 1H, J=8.5), 5.29 (br, 1H), 4.11 (t, 2H, J=6.0), 3.50 (t, 2H, J=7.0), 3.46 (m, 2H), 2.91 (m, 5H), 2.53 (t, 2H, J=6.0), 2.07 (m, 2H), 1.69 (m, 2H), 0.95 (t, 3H, J=7.0).

MS: m/z=432 (M+H).

### EXAMPLE 14

Preparation of N′-methyl-N-ethyl-N-(3-{[7-propyl-3-(trifluoromethyl)-1,2-benzisoxazol-6-yl]oxy}propyl)urea.

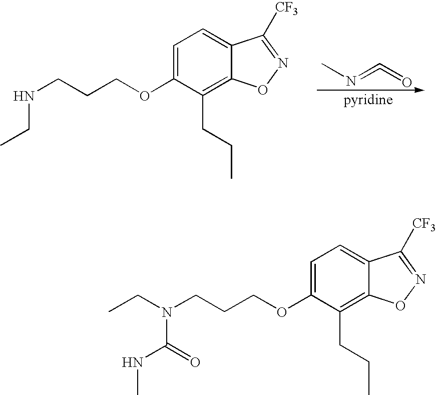

To a solution of 7-propyl-3-(trifluoromethyl)-6-(3-ethylaminopropyloxy)-1,2-benzisoxazole prepared according to the procedure of Example 7 (22.4 mg, 0.07 mmol) in pyridine (2 mL) was added methyl isocyanate (4.4 μL, 0.075 mmol). The reaction mixture was stirred at room temperature overnight. The solvent was evaporated in vacuo, and the residue was purified by chromatography on silica gel using methanol:methylene chloride (2:98) to give the title compound.

¹H NMR (CDCl₃) δ 7.54 (d, 1H, J=9.0 Hz), 7.05 (d, 1H, J=8.5), 4.49 (br, 1H), 4.13 (t, 2H, J=6.0), 3.45 (t, 2H, J=7.0), 3.25 (m, 2H), 2.91 (t, 2H, J=7.5), 2.71 (d, 3H, J=4.5), 2.08 (m, 2H), 1.70 (m, 2H), 1.15 (t, 3H, J=7.0), 0.96 (t, 3H, J=7.0).

MS: m/z=388 (M+H).

### EXAMPLE 15

Preparation of N′,N-diethyl-N-(3-{[7-propyl-3-(trifluoromethyl)-1,2-benzisoxazol-6-yl]oxy}propyl)urea.

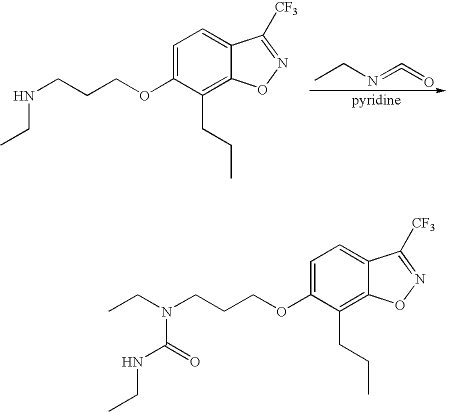

To a solution of 7-propyl-3-(trifluoromethyl)-6-(3-ethylaminopropyloxy)-1,2-benzisoxazole (23.5 mg, 0.07 mmol) in pyridine (2 mL) was added ethyl isocyanate (6.32 μL, 0.078 mmol). The reaction mixture was stirred at room temperature overnight. The solvent was evaporated in vacuo, and the residue was purified by chromatography on silica gel using methanol: methylene chloride (2:98) to give the title compound.

¹H NMR (CDCl₃) δ 7.54 (d, 1H, J=9.0 Hz), 7.05 (d, 1H, J=8.5), 4.42 (br, 1H), 4.14 (t, 2H, J=6.0), 3.45 (t, 2H, J=7.0), 3.19-3.29 (m, 4H), 2.91 (t, 2H, J=7.5), 2.08 (m, 2H), 1.16 (t, 3H, J=7.0), 1.05 (t, 3H, J=7.0), 0.97 (t, 3H, J=7.5).

MS: m/z=402 (M+H).

### EXAMPLE 16

Preparation of N′-propyl-N-ethyl-N-(3-{[7-propyl-3-(trifluoromethyl)-1,2-benzisoxazol-6-yl]oxy}propyl)urea.

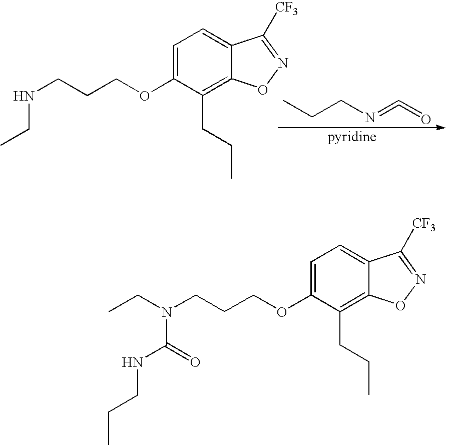

To a solution of the 7-propyl-3-(trifluoromethyl)-6-(3-ethylaminopropyloxy)-1,2-benzisoxazole (21.6 mg, 0.07 mmol) in pyridine (2 mL) was added propyl isocyanate (6.9 μL, 0.07 mmol). The reaction mixture was stirred at room temperature overnight. The solvent was evaporated in vacuo, and the residue was purified by chromatography on silica gel using methanol:methylene chloride (2:98) to give the title compound.

¹H NMR (CDCl₃) δ 7.54 (d, 1H, J=8.5 Hz), 7.05 (d, 1H, J=8.5), 4.48 (br, 1H), 4.13 (t, 2H, J=4.5), 3.45 (t, 2H, J=7.0), 3.27 (m, 2H), 3.15 (m, 2H), 2.91 (t, 2H, J=7.0), 2.07 (m, 2H), 1.70 (m, 2H), 1.45 (m, 2H), 1.16 (m, 3H), 0.96 (m,3H), 0.86 (m, 3H).

MS: m/z=416 (M+H).

### EXAMPLE 17

Preparation of N′-isopropyl-N-ethyl-N-(3-{[7-propyl-3-(trifluoromethyl)-1,2-benzisoxazol-6-yl]oxy}propyl)urea.

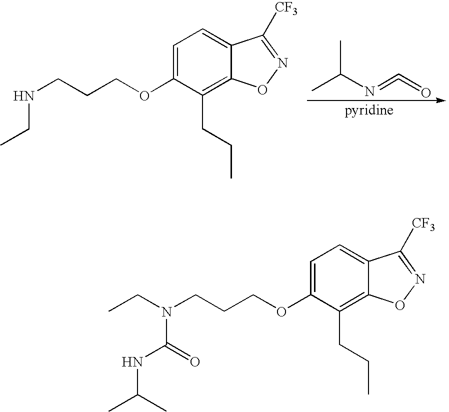

To a solution of 7-propyl-3-(trifluoromethyl)-6-(3-ethylaminopropyloxy)-1,2-benzisoxazole (21.6 mg, 0.07 mmol) in pyridine (2 mL) was added isopropyl isocyanate (7.3 μL, 0.07 mmol). The reaction mixture was stirred at room temperature overnight. The solvent was evaporated in vacuo, and the residue was purified by chromatography on silica gel using EtOAc:hexanes (1:1) to give the title compound.

¹H NMR (CDCl₃) δ 7.57 (d, 1H, J=8.5 Hz), 7.07 (d, 1H, J=8.5), 4.23 (d, 1H, J=7.0), 4.16 (t, 2H, J=6.0), 3.96 (m, 1H), 3.47 (t, 2H, J=6.5), 3.27 (m, 2H), 2.94 (t, 2H, J=7.5), 2.11(t, 2H, J=6.0), 1.75 (m, 2H), 1.18(t, 3H, J=7.0), 1.09(d, 6H, J=6.0), 1.00 (t, 3H, J=7.5).

MS: m/z=416 (M+H).

### EXAMPLE 18

Preparation of N′-phenyl-N-ethyl-N-(3-{[7-propyl-3-(trifluoromethyl)-1,2-benzisoxazol-6-yl]oxy}propyl)urea.

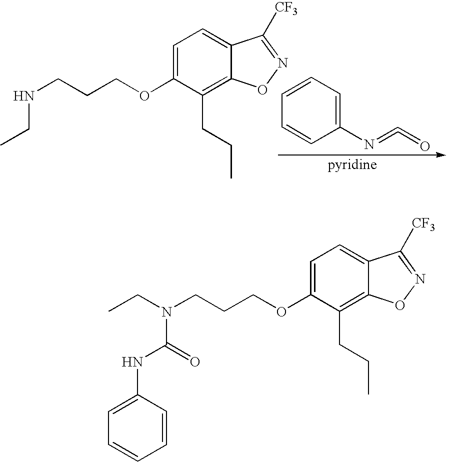

To a solution of 7-propyl-3-(trifluoromethyl)-6-(3-ethylaminopropyloxy)-1,2-benzisoxazole (24.0 mg, 0.07 mmol) in pyridine (2 mL) was added phenyl isocyanate (8.9 μL, 0.08 mmol). The reaction mixture was stirred at room temperature overnight. The solvent was evaporated in vacuo, and the residue was purified by chromatography on silica gel using EtOAc:hexanes (1:1) to give the title compound.

¹H NMR (CDCl₃) δ 7.56 (d, 1H, J=9.0 Hz), 7.20-7.31 (m, 4H), 7.08 (d, 1H, J=8.5), 7.02 (t, 1H, J=6.5), 6.54 (s, 1H), 4.20 (t, 2H, J=6.0), 3.61 (t, 2H, J=7.0), 3.44 (m, 2H), 2.96 (t, 2H, J=7.05), 2.19 (m, 2H), 1.74 (m, 2H), 1.29 (t, 3H, J=7.0), 0.99 (t, 3H, J=7.0).

MS: m/z=450 (M+H).

### EXAMPLE 19

Preparation of N′-(2-carbethoxyethyl)-N-ethyl-N-(3-{[7-propyl-3-(trifluoromethyl)-1,2-benzisoxazol-6-yl]oxy}propyl)urea and N′-(2-carboxyethyl)-N-ethyl-N-(3-}[7-propyl-3-(trifluoromethyl)-1,2-benzisoxazol-6-yl]oxy}propyl)urea.

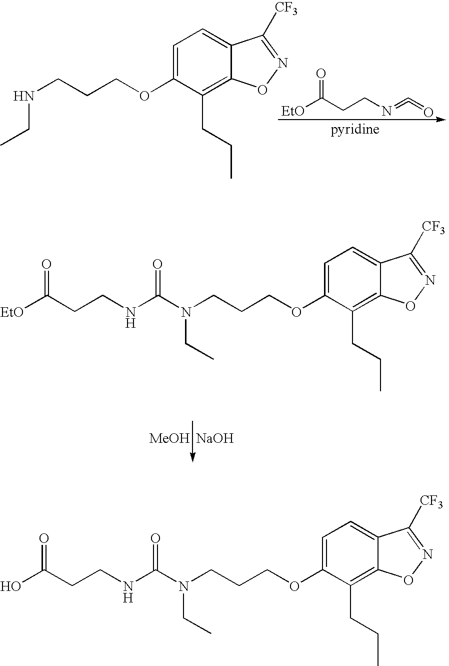

To a solution of 7-propyl-3-(trifluoromethyl)-6-(3-ethylaminopropyloxy)-1,2-benzisoxazole (30.0 mg, 0.09 mmol) in pyridine (3 mL) was added ethyl 3-isocyanatopropionate (13.4 μL, 0.1 mmol). The reaction mixture was stirred at room temperature overnight. The solvent was evaporated in vacuo, and the residue was purified by chromatography on silica gel using methanol and methylene chloride (3:97) to give the urea ester N′-(2-carbethoxyethyl)-N-ethyl-N-(3-{[7-propyl-3-(trifluoromethyl)-1,2-benzisoxazol-6-yl]oxylpropyl)urea. This urea ester (38.6 mg, 0.082 mmol) was dissolved in MeOH (1 mL) and NaOH aq (1 N, 1 mL) was added in to the solution and stirred at room temperature overnight. The reaction mixture was neutralized with 1N HCl and purified by prep-RP8 HPLC to afford N′-(2-carboxyethyl)-N-ethyl-N-(3-{[7-propyl-3-(trifluoromethyl)-1,2-benzisoxazol-6-yl]oxy}propyl)urea.

¹H NMR (CDCl₃) δ 7.59 (d, 1H, J=9.0 Hz), 7.08 (d, 1H, J=8.5), 5.25 (br, 1H,), 4.15 (t, 2H, J=5.5), 3.48 (m, 4H), 3.29 (m, 2H), 2.93 (t, 2H, J=8.0 ), 2.60 (m, 2H), 2.11 (m, 2H), 1.73 (m, 2H), 1.18 (t, 3H, J=6.5), 0.99 (t, 3H, J=7.0).

MS: m/z=446 (M+H).

### EXAMPLE 20

Preparation of N′-carbethoxymethyl-N-ethyl-N-(3-{[7-propyl-3-(trifluoromethyl)-1,2-benzisoxazol-6-yl]oxy}propyl)urea and N′-carboxymethyl-N-ethyl-N-(3-{[7-propyl-3-(trifluoromethyl)-1,2-benzisoxazol-6-yl]oxy}propyl)urea.

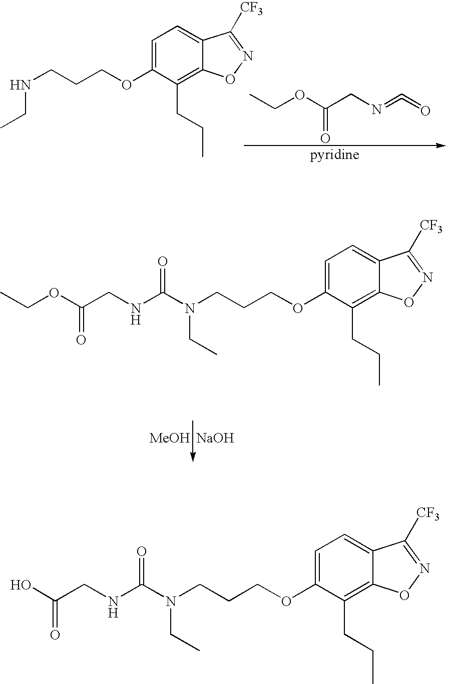

To a solution of 7-propyl-3-(trifluoromethyl)-6-(3-ethylaminopropyloxy)-1,2-benzisoxazole (30.0 mg, 0.09 mmol) in pyridine (3 mL) was added ethyl isocyanatoacetate (11.8 μL, 0.1 mmol). The reaction mixture was stirred at room temperature overnight. The solvent was evaporated in vacuo, and the residue was purified by chromatography on silica gel using methanol and methylene chloride (3:97) to give the urea ester N′-carbethoxymethyl-N-ethyl-N-(3-{[7-propyl-3-(trifluoromethyl)-1,2-benzisoxazol-6-yl]oxy}propyl)urea. The urea ester (32.7 mg, 0.07 mmol) was dissolved in MeOH (1 mL) and NaOH aq (1 N, 1 mL) was added in to the solution and stirred at room temperature overnight. The reaction mixture was neutralized with 1N HCl and purified by prep-RP8 HPLC (0.1% TFA in acetonitrile (10-100% gradient)/H₂O) to afford N′-carboxymethyl-N-ethyl-N-(3-{[7-propyl-3-(trifluoromethyl)-1,2-benzisoxazol-6-yl]oxy}propyl)urea.

¹H NMR (CDCl₃) δ 7.59 (d, 1H, J=9.0 Hz), 7.08 (d, 1H, J=9.0), 5.29 (m, 1H), 4.18 (t, 2H, J=5.5), 3.98 (s, 2H), 3.53 (t, 2H, J=7.0), 3.35 (m, 2H),), 2.94 (t, 2H, J=7.5 ), 2.15 (t, 2H, J=6.0), 1.71 (m, 2H), 1.23 (t, 3H, J=7.0 ), 1.01 (t, 3H, J=7.0).

MS: m/z=432 (M+H).

### EXAMPLE 21

Preparation of N′-(1(S)-carboxyethyl)-N-methyl-N-(3-{[7-propyl-3-(trifluoromethyl)-1,2-benzisoxazol-6-yl]oxy}propyl)urea.

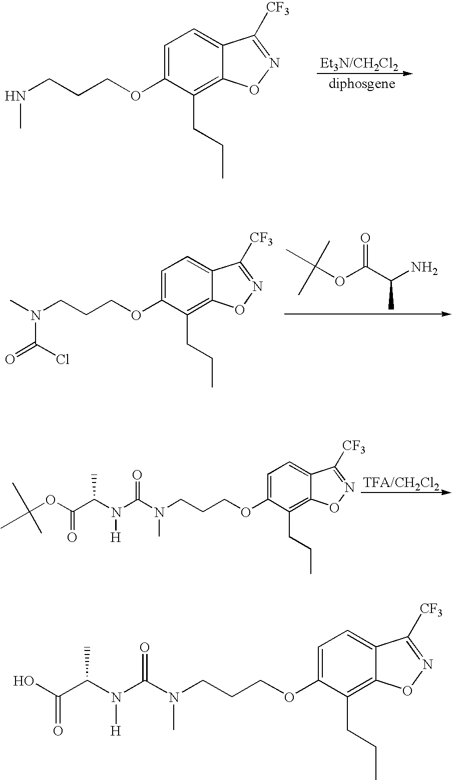

At 0° C., a solution of 7-propyl-3-(trifluoromethyl)-6-(3-methylaminopropyloxy)-1,2-benzisoxazole (70.3 mg, 0.2225 mmol) in methylene chloride (1 mL) was added to a solution of diphosgene (13.9 μL, 0.1112 mmol) in methylene chloride (2 mL), followed by addition of Et₃N (46.6 μL, 0.3336 mmol), then stirred at 0° C. for 2 hours. The t-butyl amino ester (161.6 mg, 1.113 mmol) in methylene chloride (2 mL) was then added into the above reaction mixture and stirred at room temperature overnight. The solvent was evaporated in vacuo, and the residue was purified by chromatography on silica gel using EtOAc: hexanes (1:1) to give the urea ester compound. The urea ester (64.0 mg, 0.13 mmol) was dissolved in methylene chloride (1 mL), followed by adding TFA ( 0.4 mL) and stirred at room temperature overnight. The solvent was evaporated in vacuo, and was purified by prep-RP8 HPLC (0.1% TFA in acetonitrile (10-100% gradient)/H₂O) to give the title compound.

¹H NMR (CDCl₃) δ 7.59 (d, 1H, J=8.5 Hz), 7.09 (d, 1H, J=9.0),4.90 (m, 1H), 4.38 (m, 1H), 4.18 (m, 2H), 3.58 (m, 2H), 3.00 (s, 3H), 2.95 (t, 2H, J=7.5), 2.14 (m, 2H), 1.74 (m, 2H), 1.40 (d, 3H, J=7.0), 1.00 (t, 3H, J=7.0).

MS: m/z=432(M+H).

### EXAMPLE 22

Preparation of racemic N′-methyl-N′-(1-carboxyethyl)-N-methyl-N-(3-{[7-propyl-3-(trifluoromethyl)-1,2-benzisoxazol-6-yl]oxy}propyl)urea, sodium salt.

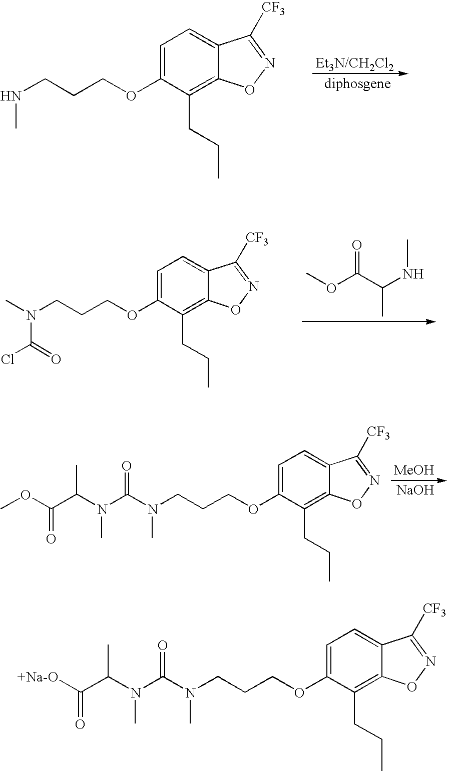

At 0 ° C., a solution of 7-propyl-3-(trifluoromethyl)-6-(3-methylaminopropyloxy)-1,2-benzisoxazole (31.2 mg, 0.099 mmol) in methylene chloride (0.5 mL) was added to a solution of diphosgene (6.2 μL, 0.049 mmol) in methylene chloride (1 mL), followed by addition of Et₃N (20.6 μL, 0.1481 mmol), then stirred at 0° C. for 2 hours. The amino ester (58.0 mg, 0.495 mmol) in methylene chloride (2 mL) was then added into the above reaction mixture and stirred at room temperature overnight. The solvent was evaporated in vacuo, and the residue was purified by chromatography on silica gel using EtOAc:hexanes (1:1) to give the urea ester. The urea ester (31.0 mg, 0.0675 mmol) was dissolved in methanol (0.3 mL) and NaOH aq ( 68 μL, 0.068 mmol) was added and stirred at room temperature overnight. The solvent was evaporated in vacuo to afford the title compound.

¹H NMR (CDCl₃) δ 7.65 (m, 1H), 7.29 (m, 1H), 4.90 (m, 1H), 4.27 (m, 1H), 4.20 (m, 2H), 3.44 (m, 2H), 2.91(m, 2H), 2.89 (s, 3H), 2.82 (s, 3H), 2.14 (m, 2H), 1.74 (m, 2H), 1.34 (d, 3H, J=7.0), 0.97 (m, 3H).

MS: m/z=468(M+H).

### EXAMPLE 23

Preparation of racemic N′-(1-carboxyethyl)-N-methyl-N-(3-{[7-propyl-3-(trifluoromethyl)-1,2-benzisoxazol-6-yl]oxy}propyl)urea.

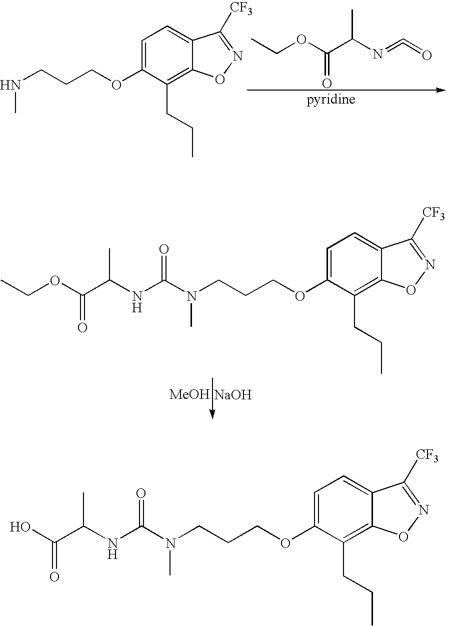

To a solution of the 7-propyl-3-(trifluoromethyl)-6-(3-methylaminopropyloxy)-1,2-benzisoxazole(41.5 mg, 0.13 mmol) in pyridine (2 mL) was added ethyl 2-isocyanatopropionate (20.0 μL, 0.15 mmol). The reaction mixture was stirred at room temperature overnight. The solvent was evaporated in vacuo, and the residue was purified by chromatography on silica gel using methanol and methylene chloride (2:98) to give the urea ester. The urea ester (48.4 mg, 0.11 mmol) was dissolved in MeOH (0.6 mL) and NaOH aq (1 N, 0.43 mL) was added in to the solution and stirred at room temperature overnight. The reaction mixture was neutralized with 1N HCl and purified by prep-RP8 HPLC (0.1% TFA in acetonitrile (10-100% gradient)/H₂O) to afford the title compound.

¹H NMR (CDCl₃) δ 9.18 (br, 1H), 7.59 (d, 1H, J=8.5 Hz), 7.09 (d, 1H, J=9.0), 5.16 (br, 1H), 4.38 (m, 1H), 4.18 (m, 2H), 3.58 (m, 2H), 3.00 (s, 3H), 2.95 (t, 2H, J=7.5), 2.14 (m, 2H), 1.40 (d, 3H, J=7.0), 1.00 (t, 3H, J=7.0).

MS: m/z=432 (M+H).

### EXAMPLE 24

Preparation of racemic N′-(2-carboxypropyl)-N-methyl-N-(3-{[7-propyl-3-(trifluoromethyl)-1,2-benzisoxazol-6-yl]oxy}propyl)urea.

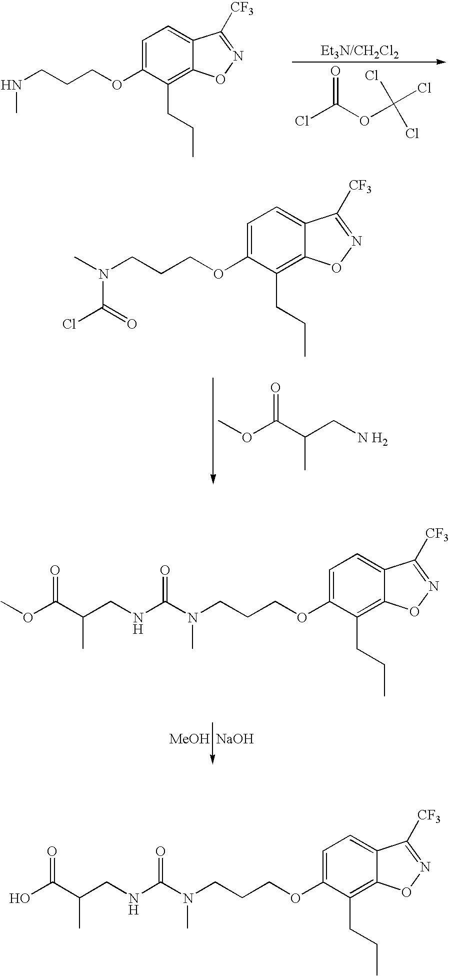

At 0° C., a solution of the 7-propyl-3-(trifluoromethyl)-6-(3-methylaminopropyloxy)-1,2-benzisoxazole(178.0 mg, 0.259 mmol) in methylene chloride (1.0 mL) was added to a solution of diphosgene (21 μL, 0.168 mmol) in methylene chloride (0.75 μL), followed by addition of Et₃N (70.5 μL, 1.01 mmol), then stirred at 0° C. for 2 hours. The amino ester (150.2 mg, 1.295 mmol) in methylene chloride (2 mL) was then added into the above reaction mixture and stirred at room temperature overnight. The solvent was evaporated in vacuo, and the residue was purified by chromatography on silica gel using EtOAc:hexanes (1:1) to give the urea ester. The urea ester (25.4 mg, 0.055 mmol) was dissolved in methanol (0.5 mL) and NaOH aq (0.23 mL, 0.23 mmole) was added and stirred at room temperature overnight. The solvent was evaporated in vacuo, the residue neutralized with 1N HCl and purified by prep-RP8 HPLC (0.1% TFA in acetonitrile (10-100% gradient)/H₂O) to afford the title compound.

¹H NMR (CDCl₃) δ 7.58 (d, 1H, J=8.5 Hz), 7.09 (d, 1H, J=9.0), 5.17 (br, 1H), 4.14 (t, 2H, J=6.5), 3.53 (t, 2H, J=7.0), 3.35 (m, 2H), 2.94 (s, 3H), 2.94 (t, 2H, J=7.0), 2.71 (m, 1H), 2.12 (m, 2H), 1.74 (m, 2H), 1.19 (d, 3H, J=7.0), 0.99 (d, 3H, J=7.5).

MS: m/z=446(M+H).

### EXAMPLE 25

Preparation of N′-(2-pyridyl)-N-methyl-N-(3-{[7-propyl-3-(trifluoromethyl)-1,2-benzisoxazol-6-yl]oxy}propyl)urea.

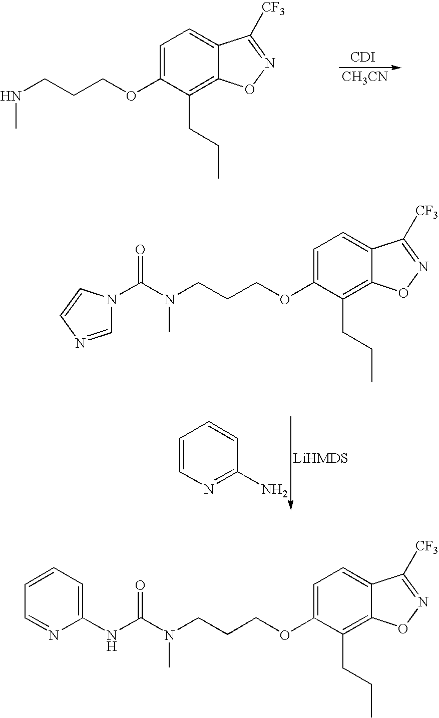

At 0° C., CDI ( 2.46 g, 15.19 mmol) was added to a solution of the 7-propyl-3-(trifluoromethyl)-6-(3-methylaminopropyloxy)-1,2-benzisoxazole(2.4 g, 7.59 mmol) in acetonitrile (120 mL), then stirred at RT for 2 hours. The reaction mixture was then poured into ice water (50 mL) and diluted with ethyl acetate (50 mL). The aqueous layer was extracted with ethyl acetate and the combined ethyl acetate extracts were washed with water (2×50 mL) and with brine (50 mL), dried over Na₂SO₄, filtered and the solvent was evaporated in vacuo. At 0° C., LiHMDS (1 M hexanes, 11.4 mL, 11.4 mmol) was added to 2-amino pyridine (0.7 g, 7.22 mmol) in THF (20 mL) and stirred for 30 min. At 0° C., the resulting mixture was added to the acyl imidazole (prepared above) in THF (60 mL) and stirred at RT overnight. Another addition of LiHMDS (1 M hexanes, 7.8 mL, 7.8 mmol) was made to the reaction mixture at 0° C. and stirred at RT for 5 hours. NH₄Cl (sat'd aq) (150 mL) was then added to the reaction mixture and the resulting mixture diluted with ethyl acetate (100 mL). The aqueous layer was extracted with ethyl acetate (2×100 mL) and the combined ethyl acetate extracts were washed with H₂O (1×100 mL), NH₄Cl (sat'd aq, 1×100 mL), brine (1×100 mL), then dried over Na₂SO₄ and filtered. The solvent was evaporated in vacuo, and the residue was purified by chromatography on silica gel using acetone:hexanes (2.5:7.5) to give the title compound.

¹H NMR (CDCl₃) δ 8.17 (m, 1H), 8.04 (d, 1H, J=8.0 Hz), 7.65 (t, 1H, J=6.0), 7.57 (d, 1H, J=9.0), 7.22 (br, 1H), 7.08 (d, 1H, J=9.0), 6.95 (m, 1H), 4.21 (t, 2H, J=6.0), 3.67 (t, 2H, J=7.0), 3.12 (s, 3H), 2.97 (t, 2H, J=7.0), 2.21 (m 2H), 1.77 (m, 2H), 1.00 (t, 3H, J=7.5).

MS: m/z=437 (M+H).

### EXAMPLE 26

Preparation of N′-(3-pyridyl)-N-methyl-N-(3-{[7-propyl-3-(trifluoromethyl)-1,2-benzisoxazol-6-yl]oxy}propyl)urea.

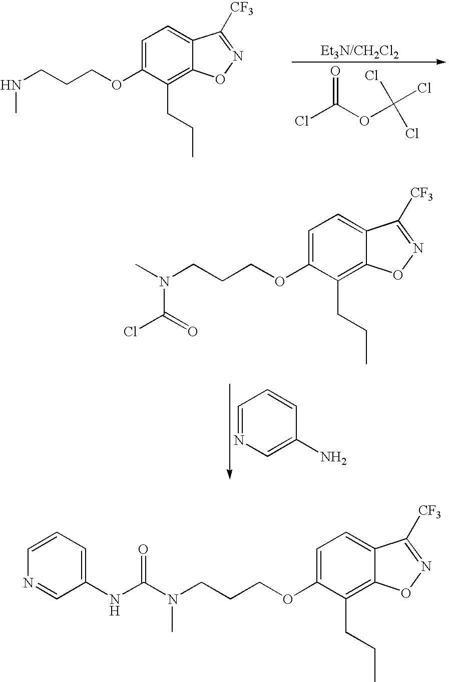

At 0° C., a solution of the 7-propyl-3-(trifluoromethyl)-6-(3-methylaminopropyloxy)-1,2-benzisoxazole(85.7 mg, 0.2712 mmol) in methylene chloride (1.0 mL) was added to a solution of diphosgene (44.0 μL, 0.049 mmol) in methylene chloride (2 mL), followed by addition of Et₃N (148 μL, 1.06 mmol), then stirred at 0° C. for 2 hours. The amino pyridine (71.0 mg, 0.75 mmol) in methylene chloride (4 mL) was then added into the above reaction mixture and stirred at room temperature overnight. The solvent was evaporated in vacuo, and the residue was purified by chromatography on silica gel using MeOH:methylene chloride (1:19) to give the title compound.

¹H NMR (CDCl₃) δ 8.37 (s, 1H), 8.27 (m, 1H), 7.94 (d, 1H, J=5.5), 7.59 (d, 1H, J=9.0), 7.21 (m, 1H), 7.09 (d, 1H, J=8.5), 6.60 (s, 1H), 4.22 (t, 2H, J=6.0), 3.67 (t, 2H, J=7.0), 3.12 (s, 3H), 2.97 (t, 2H, J=7.5), 2.20 (t, 2H, J=6.5), 1.76 (m, 2H), 0.99 (t, 3H, J=7.0).

MS: m/z=437 (M+H).

### EXAMPLE 27

Preparation of N′-(4-pyridyl)-N-methyl-N-(3-{[7-propyl-3-(trifluoromethyl)-1,2-benzisoxazol-6-yl]oxy}propyl)urea.

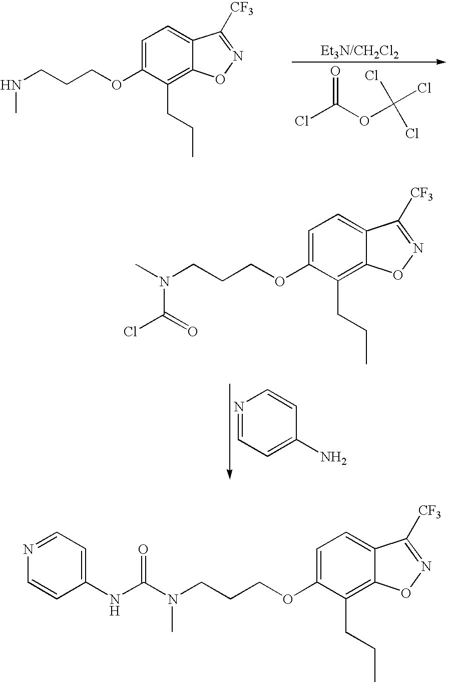

At 0° C., a solution of the 7-propyl-3-(trifluoromethyl)-6-(3-methylaminopropyloxy)-1,2-benzisoxazole(85.7 mg, 0.2712 mmol) in methylene chloride (1.0 mL) was added to a solution of diphosgene (44.0 μL, 0.049 mmol) in methylene chloride (2 mL), followed by addition of Et₃N (148 μL, 1.06 mmol), then stirred at 0° C. for 2 hours. The 4-amino pyridine (71.0 mg, 0.75 mmol) in methylene chloride (4 mL) was then added into the above reaction mixture and stirred at room temperature overnight. The solvent was evaporated in vacuo, and the residue was purified by chromatography on silica gel using MeOH:methylene chloride (1:19) to give the title compound.

¹H NMR (CDCl₃) δ 8.38 (m, 2H), 7.59 (d, 1H, J=9.0 Hz), 7.28 (m, 2H), 7.10 (d, 1H, J=9.0), 6.80 (s, 1H), 4.21 (t, 2H, J=5.5), 3.66 (t, 2H, J=7.0), 3.12 (s, 3H), 2.97 (t, 2H, J=7.5), 2.20 (m, 2H), 1.76 (m, 2H), 1.0 (t, 3H, J=7.5).

MS: m/z=437 (M+H).

### EXAMPLE 28

Preparation of N′-ethyl-N′-methyl-N-methyl-N-(3-{[7-propyl-3-(trifluoromethyl)-1,2-benzisoxazol-6-yl]oxy}propyl)urea.

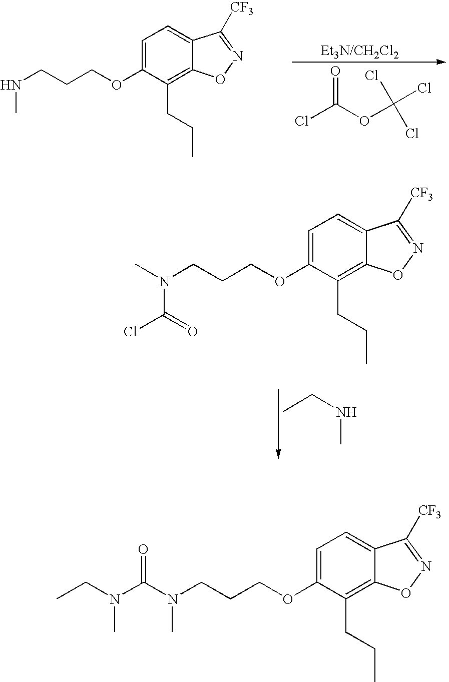

At 0° C., a solution of the 7-propyl-3-(trifluoromethyl)-6-(3-methylaminopropyloxy)-1,2-benzisoxazole(70.2 mg, 0.222 mmol) in methylene chloride (0.5 mL) was added to a solution of diphosgene (18.5 μL, 0.148 mmol) in methylene chloride (1 mL), followed by addition of Et₃N (62 μL, 0.4443 mmol), then stirred at 0° C. for 2 hours. The indicated amine (66.0 mg, 1.11 mmol) in methylene chloride (0.5 mL) was then added into the above reaction mixture and stirred at room temperature overnight. The solvent was evaporated in vacuo, and the residue was purified by chromatography on silica gel using MeOH:methylene chloride (1:19) to give the title compound.

¹H NMR (CDCl₃) δ 7.53 (d, 1H, J=9.0 Hz), 7.05 (d, 1H, J=8.5), 4.12 (m, 2H), 3.48 (t, 2H, J=7.0), 3.21 (m, 2H), 2.90 (m, 8H), 2.07 (m, 2H), 1.70 (m, 2H), 1.04 (t, 3H, J=7.0), 0.95 (m, 3H).

MS: m/z=402 (M+H).

### EXAMPLE 29

Preparation of N′,N′-dimethyl-N-methyl-N-(3-{[7-propyl-3-(trifluoromethyl)-1,2-benzisoxazol-6-yl]oxy}propyl)urea.

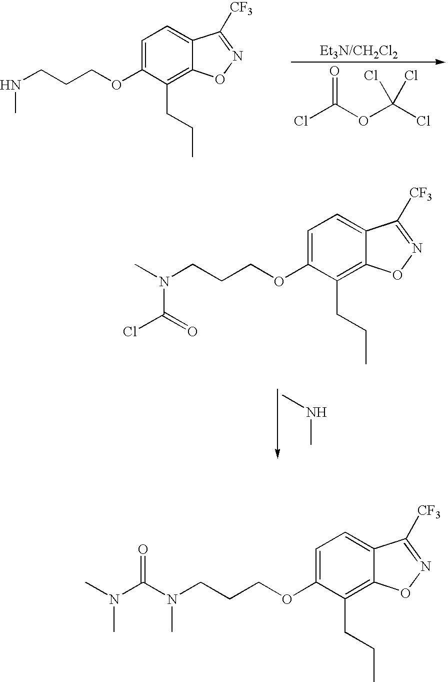

At 0° C., a solution of the 7-propyl-3-(trifluoromethyl)-6-(3-methylaminopropyloxy)-1,2-benzisoxazole(49.1 mg, 0.155 mmol) in methylene chloride (0.5 mL) was added to a solution of diphosgene (13.3 μL, 0.107 mmol) in methylene chloride (1 mL), followed by addition of Et₃N (45 μL, 0.32 mmol), then stirred at 0° C. for 2 hours. The dimethylamine (42.0 mg, 0.94 mmol) in methylene chloride (0.5 mL) was then added into the above reaction mixture and stirred at room temperature overnight. The solvent was evaporated in vacuo, and the residue was purified by chromatography on silica gel using MeOH:methylene chloride (3:97) to give the title compound.

¹H NMR δ 7.55 (d, 1H, J=8.5 Hz), 7.05 (d, 1H, J=9.0), 4.13 (t, 2H, J=6.0), 3.48 (t, 2H, J=7.0), 2.91 (m, 8H), 2.77 (m, 3H), 2.08 (m, 2H), 1.70 (m, 2H), 0.94 (m, 3H).

MS: m/z=388 (M+H).

### EXAMPLE 30

Preparation of N′-(2-pyridyl)-N′-methyl-N-methyl-N-(3-{[7-propyl-3-(trifluoromethyl)-1,2-benzisoxazol-6-yl]oxy}propyl)urea.

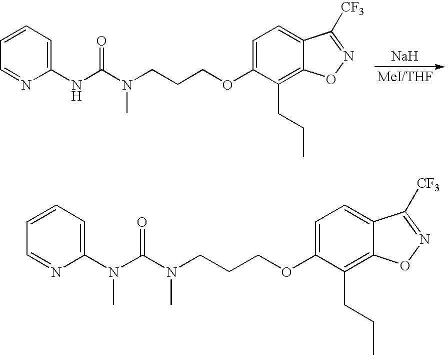

NaH (0.9 mg, 0.022 mmol) was added to the pyridyl urea, prepared according to the procedure of Example 25, (8.0 mg, 0.018 mmol) in THF (1.0 mL), followed by addition of MeI (1.8 μL, 0.028 mmol) and the mixture stirred at RT for 4 hours. The solvent was evaporated in vacuo, and the residue was purified by chromatography on silica gel using MeOH:methylene chloride (3:97) to give the title compound.

¹H NMR (CDCl₃) δ 8.30 (m, 1H), 7.56 (m, 2H), 7.08 (d, 1H, J=9.0 Hz), 6.88 (m, 2H), 4.21 (t, 2H, J=6.0), 3.67 (t, 2H, J=7.0), 3.31 (s, 3H), 2.97 (t, 2H, J=7.0), 2.84 (s, 3H), 1.77 (m, 2H), 1.00 (t, 3H, J=7.5).

MS: m/z=451 (M+H).

### EXAMPLE 31

Preparation of N-methyl-N-(3-{[7-propyl-3-(trifluoromethyl)-1,2-benzisoxazol-6-yl]oxy}propyl)urea.

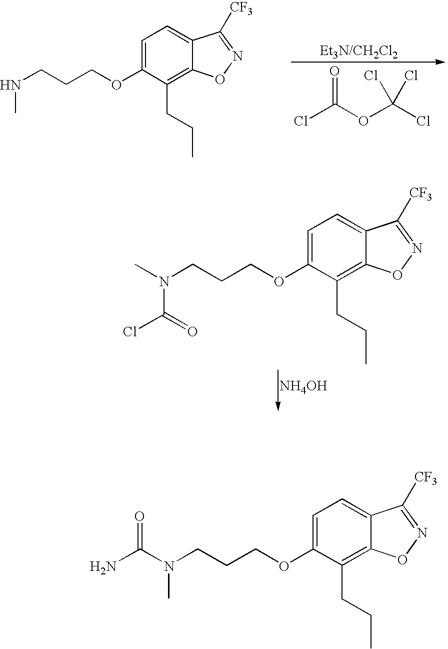

At 0° C., a solution of the 7-propyl-3-(trifluoromethyl)-6-(3-methylaminopropyloxy)-1,2-benzisoxazole(70.9 mg, 0.22 mmol) in methylene chloride (0.5 mL) was added to a solution of diphosgene (19.2 μL, 0.15 mmol) in methylene chloride (1 mL), followed by addition of Et₃N (64 μL, 0.46 mmol), then stirred at 0° C. for 2 hours. Aqueous ammonium hydroxide (approximately 6M, 0.15 mL, 0.9 mmol) in methylene chloride (0.5 mL) was then added into the above reaction mixture and stirred at room temperature overnight. The solvent was evaporated in vacuo, and the residue was purified by chromatography on silica gel using MeOH:methylene chloride (1:19) to give the title compound.

¹H NMR (CDCl₃) δ 7.55 (d, 1H, J=9.0 Hz), 7.05 (d, 1H, J=9.0), 4.86 (s, 2H), 4.15 (t, 2H, J=6.0), 3.51 (t, 2H, J=7.0), 2.95 (s, 3H), 2.91 (t, 2H, J=7.5), 2.11 (m, 2H), 1.71 (m, 2H), 1.70 (m, 2H), 0.97 (t, 2H, J=7.0).

MS: m/z=360 (M+H).

### EXAMPLE 32

Preparation of N′-methyl-N′-(4-pyridyl)-N-methyl-N-(3-{[7-propyl-3-(trifluoromethyl)-1,2-benzisoxazol-6-yl]oxy}propyl)urea.

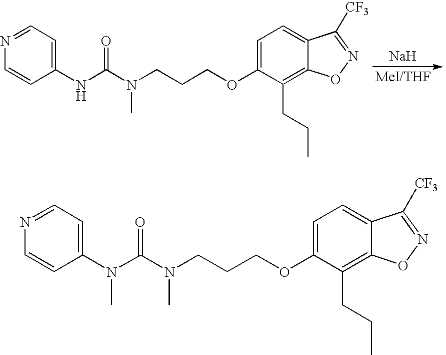

NaH (1.4 mg, 0.034 mmol) was added to the pyridyl urea, prepared according to the procedure of Example 27, (12.1 mg, 0.028 mmol) in THF (1.0 mL) followed by addition of MeI (2.6 μL, 0.04 mmol) and the mixture stirred at RT for 4 hours. The solvent was evaporated in vacuo, and the residue was purified by chromatography on silica gel using MeOH: methylene chloride (3:97) to give the title compound.

¹H NMR (CDCl₃) δ 8.38 (m, 2H), 7.59 (d, 1H, J=9.0 Hz), 7.28 (m, 2H), 7.10 (d, 1H, J=9.0), 6.80 (s, 1H), 4.21 (t, 2H, J=5.5), 3.66 (t, 2H, J=7.0), 3.23 (s, 3H), 2.97 (t, 2H, J=7.5), 2.88 (s, 3H), 2.20 (m, 2H), 1.76 (m, 2H), 1.0 (t, 3H, J=7.5).

MS: m/z=451 (M+H).

### EXAMPLE 33

Preparation of N′-(5-tetrazolyl)-N-methyl-N-(3-{[7-propyl-3-(trifluoromethyl)-1,2-benzisoxazol-6-yl]oxy}propyl)urea.

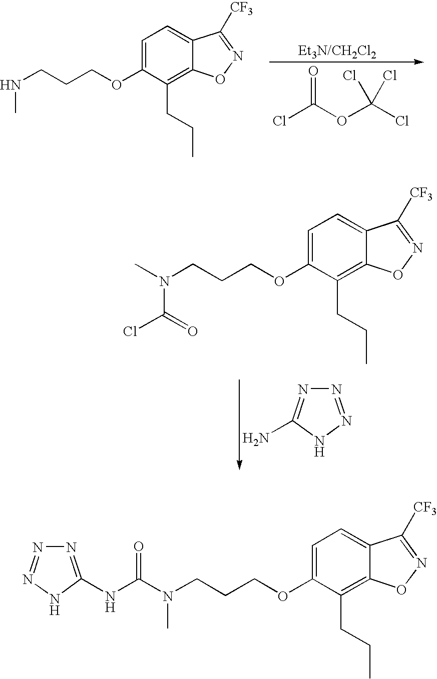

At 0° C., a solution of the 7-propyl-3-(trifluoromethyl)-6-(3-methylaminopropyloxy)-1,2-benzisoxazole(74.3 mg, 0.24 mmol) in methylene chloride (0.5 mL) was added to a solution of diphosgene (20.1 μL, 0.16 mmol) in methylene chloride (1 mL), follow by addition of Et₃N (68 μL, 0.49 mmol), then stirred at 0° C. for 2 hours. The indicated amine (48.6 mg, 0.48 mmol) in methylene chloride (2 mL) and DMF (0.1 mL) and H₂O (0.1 mL) was then added into the above reaction mixture, followed by addition of DMAP (5.89 mg, 0.048 mmol) and the reaction mixture was stirred at RT overnight. The solvent was evaporated in vacuo, and the residue was purified by chromatography on silica gel using MeOH:methylene chloride (1:19) to give the title compound.

¹H NMR (DMSO) δ 10.56 (s, 1H), 7.76 (d, 1H, J=8.5 Hz), 7.35 (d, 1H, J=9.0), 4.21 (t, 2H, J=6.0), 3.56 (t 2H, J=7.0) 3.30 (s, 1H) 3.02 (s, 3H), 2.86 (t, 2H, J=8.0), 2.07 (m, 2H), 1.63 (m, 2H), 0.89 (t, 3H, J=7.5).

MS: m/z=428 (M+H).

### EXAMPLE 34

Preparation of N′-(2-pyrimidinyl)-N-methyl-N-(3-{[7-propyl-3-(trifluoromethyl)-1,2-benzisoxazol-6-yl]oxy}propyl)urea.

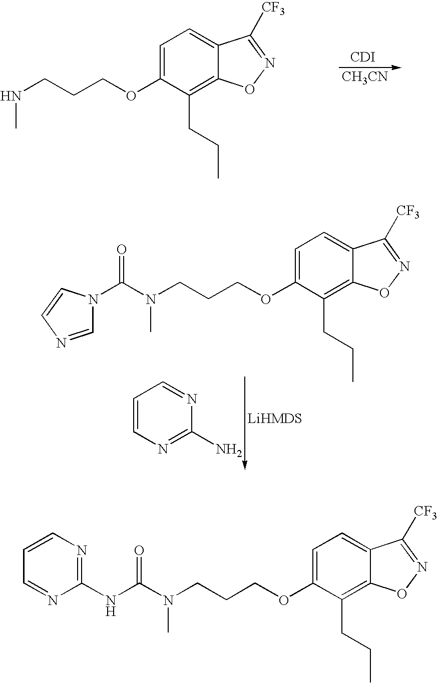

At 0° C., CDI (54.7 mg, 0.338 mmol) was added to the 7-propyl-3-(trifluoromethyl)-6-(3-methylaminopropyloxy)-1,2-benzisoxazole(53.3 mg, 0.169 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), then dried over Na₂SO₄, filtered and the solvent evaporated in vacuo. At 0° C., LiHMDS (1 M hexanes, 0.26 mL, 0.26 mmol) was added to 2-amino pyrimidine (16 mg, 0.168 mmol) in THF (1 mL) and stirred for 30 min. At 0° C., the mixture was added into the acyl imidazole (prepared above) in THF (2 mL) and stirred at RT overnight. Another addition of LiHMDS (1 M hexanes, 0.17 mL, 0.17 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL) and the combined ethyl acetate was washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 8.42 (br, 2H), 7.60 (d, 1H, J=9.0 Hz), 7.27 (d, 1H, J=9.0), 6.91 (br, 1H), 4.62 (s, 1H), 4.23 (t, 2H, J=6.0), 3.71 (t, 2H, J=6.5), 3.10 (s, 3H), 2.92 (t, 2H, J=7.5), 2.19 (m, 2H), 1.71 (m 2H), 0.95 (t, 3H, J=7.5).

MS: m/z=438 (M+H).

### EXAMPLE 35

Preparation of N′-(2-pyrazinyl)-N-methyl-N-(3-{[7-propyl-3-(trifluoromethyl)-1,2-benzisoxazol-6-yl]oxy}propyl)urea.

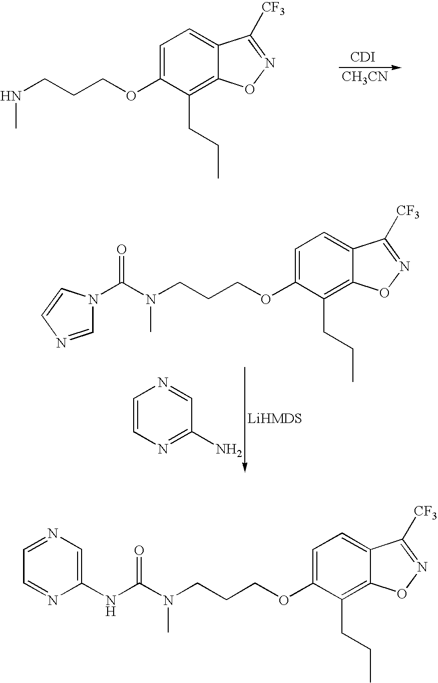

At 0° C., CDI (54.7 mg, 0.338 mmol) was added to the 7-propyl-3-(trifluoromethyl)-6-(3-methylaminopropyloxy)-1,2-benzisoxazole(53.3 mg, 0.169 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate was washed with water (2×5 mL) and with brine (5 mL), then dried over Na₂SO₄, filtered and the solvent was evaporated in vacuo. At 0° C. LiHMDS (1 M hexanes, 0.26 mL, 0.26 mmol) was added to 2-amino pyrazine (16 mg, 0.168 mmol) in THP (1 mL) and stirred for 30 min. At 0° C. this mixture was added to the acyl imidazole (prepared above) in THF (2 mL) and stirred at RT overnight. Another addition of LiHMDS (1 M hexanes, 0.17 mL, 0.17 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL) and the combined ethyl acetate extracts were washed with H₂O (1×15 mL), NH₄Cl (sat'd aq, 1×10 mL), brine (1×5 mL dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 9.43 (s, 1H), 8.25 (m, 1H), 8.09 (s, 1H), 7.58 (d, 1H, J=9.0 Hz), 7.39 (s, 1H), 7.09 (d, 1H, J=9.0), 4.22 (t, 2H, J=6.0), 3.71 (t, 2H, J=6.5), 3.10 (s, 3H), 2.92 (t, 2H, J=7.5), 2.19 (m, 2H), 1.71 (m 2H), 0.95 (t, 3H, J=7.5).

MS: m/z=438 (M+H).

### EXAMPLE 36

Preparation of N′-(6-methyl-2-pyridyl)-N-methyl-N-(3-{[7-propyl-3-(trifluoromethyl)-1,2-benzisoxazol-6-yl]oxy}propyl)urea.

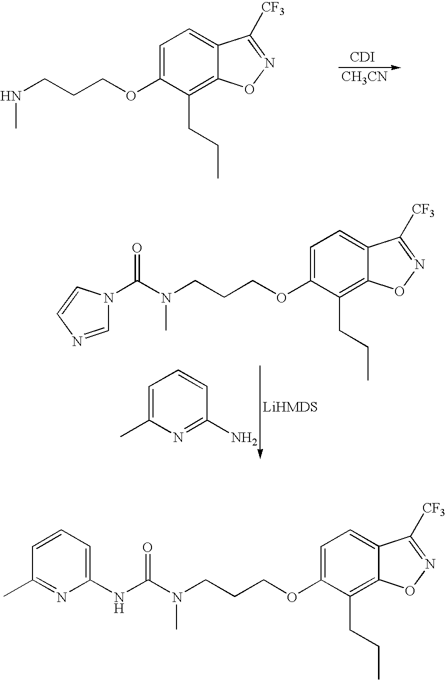

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 μL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), dried over Na₂SO₄, filtered and the solvent was evaporated in vacuo. At 0° C. LiHMDS (1 M hexanes, 0.29 mL, 0.29 mmol) was added to 6-methyl-2-amino pyridine (20.4 mg, 0.18 mmol) in THF (1 mL) and stirred for 30 min. At 0° C. this mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL). The combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 7.82 (d, 1H, J=8.0 Hz), 7.55 (m, 2H), 7.16 (s, 1H), 7.08 (d, 1H, J=9.0), 6.79 (d, 1H, J=7.5), 4.20 (t, 2H, J=6.0), 3.67 (t, 2H, J=7.0), 3.11 (s, 3H), 2.97 (t, 2H, J=7.0), 2.37 (s, 3H), 2.21 (m 2H), 1.77 (m, 2H), 1.00 (t, 3H, J=7.5).

MS: m/z=451 (M+H).

### EXAMPLE 37

Preparation of N′-(4-pyrimidinyl)-N-methyl-N-(3-{[7-propyl-3-(trifluoromethyl)-1,2-benzisoxazol-6-yl]oxy}propyl)urea.

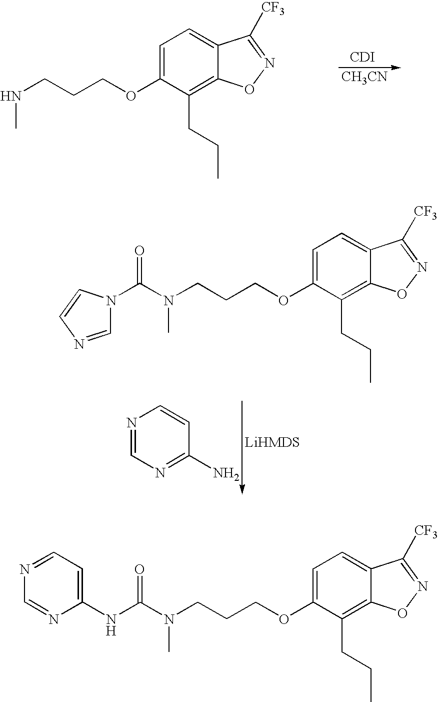

At 0° C., CDI (54.7 mg, 0.338 mmol) was added to the 7-propyl-3-(trifluoromethyl)-6-(3-methylaminopropyloxy)-1,2-benzisoxazole(53.3 mg, 0.169 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), dried over Na₂SO₄, filtered and the solvent was evaporated in vacuo. At 0° C. LiHMDS (1 M hexanes, 0.26 mL, 0.26 mmol) was added to 4-amino pyrimidine (16 mg, 0.168 mmol) in THF (1 mL) and stirred for 30 min. At 0° C. the mixture was added to the acyl imidazole, prepared above, in THF (2 mL) and stirred at RT overnight. Another addition of LiHMDS (1 M hexanes, 0.17 mL, 0.17 mmol) was made to the reaction mixture at 0° C. and stirred at RT for 5 hours. NH₄Cl (sat'd aq, 5 mL) was then added into the reaction mixture and diluted with ethyl acetate (5 mL). The aqueous layer was extracted with ethyl acetate (2×5 mL) and the combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 8.72 (s,1H), 8.52 (d, 1H, J=5.5 Hz), 8.03 (d, 1H, J=5.5), 7.58 (d, 1H, J=9.0), 7.42 (s,1H), 7.09 (d, 1H, J=9.0), 4.22 (t, 2H, J=6.0), 3.71 (t, 2H, J=6.5), 3.10 (s, 3H), 2.92 (t, 2H, J=7.5), 2.19 (m, 2H), 1.71 (m 2H), 0.95 (t, 3H, J=7.5).

MS: m/z=438 (M+H).

### EXAMPLE 38

Preparation of N′-(4,6-dimethyl-2-pyridyl)-N-methyl-N-(3-{[7-propyl-3-(trifluoromethyl)-1,2-benzisoxazol-6-yl]oxy}propyl)urea.

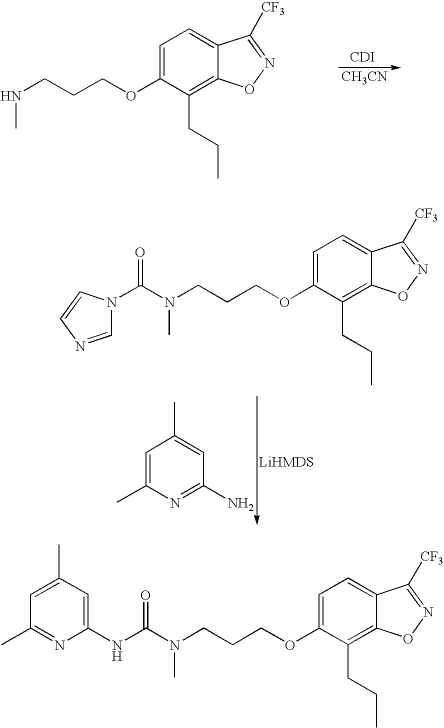

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), dried over Na₂SO₄, filtered and the solvent was evaporated in vacuo. At 0° C., LiHMDS (1 M hexanes, 0.29 mL, 0.29 mmol) was added to 4,6-dimethyl-2-amino pyridine (22.0 mg, 0.18 mmol) in THF (1 mL) and stirred for 30 min. At 0° C., the mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL). The aqueous layer was extracted with ethyl acetate (2×5 mL) and the combined ethyl acetate extracts were washed with H₂O (1×15 mL), NH4Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 7.67 (s, 1H), 7.55 (d, 1H, J=8.5 Hz), 7.16 (s, 1H), 7.07 (d, 1H, J=9.0), 6.60 (m, 1H), 4.20 (t, 2H, J=6.0), 3.67 (t, 2H, J=7.0), 2.32 (s, 3H), 2.28 (s, 3H), 2.97 (t, 2H, J=7.0), 2.37 (s, 3H), 2.21 (m 2H), 1.77 (m, 2H), 1.00 (t, 3H, J=7.5).

MS: m/z=465(M+H).

### EXAMPLE 39

Preparation of N′-(5-methyl-3-oxazolyl)-N-methyl-N-(3-{[7-propyl-3-(trifluoromethyl)-1,2-benzisoxazol-6-yl]oxy}propyl)urea.

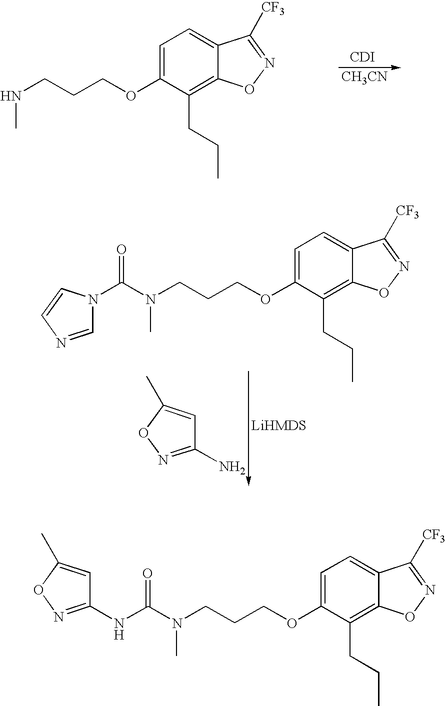

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), dried over Na₂SO₄, filtered and the solvent was evaporated in vacuo. At 0° C. LiHMDS (1 M hexanes, 0.29 mL, 0.29 mmol) was added to the indicated oxazolyl amine (17.7 mg, 0.18 mmol) in THF (1 mL) and stirred for 30 min. At 0° C., the mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL). The aqueous layer was extracted with ethyl acetate (2×5 mL) and the combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 7.59 (d, 1H, J=8.5 Hz), 7.40 (s, 1H), 7.08 (d, 1H, J=9.0), 6.62 (s, 1H), 4.19 (t, 2H, J=6.0), 3.64 (t, 2H, J=7.5), 3.10 (s, 3H), 2.96 (t, 2H, J=7.5), 2.39 (s, 3H), 218 (m, 2H), 1.76 (m, 2H), 1.00 (t, 3H, J=7.0).

MS: m/z=441 (M+H).

### EXAMPLE 40

Preparation of N′-(4-methyl-2-pyridyl)-N-methyl-N-(3-{[7-propyl-3-(trifluoromethyl)-1,2-benzisoxazol-6-yl]oxy}propyl)urea.

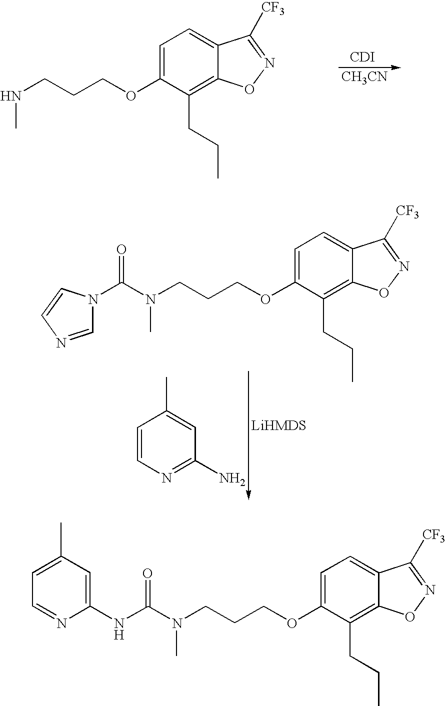

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), dried over Na₂SO₄, filtered and the solvent was evaporated in vacuo. At 0° C., LiHMDS (1 M hexanes, 0.29 mL, 0.29 mmol) was added to the indicated amino pyridine (19.5 mg, 0.18 mmol) in THF (1 mL) and stirred for 30 min. At 0° C., the mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL) and the combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 8.02 (d, 1H, J=5.0 Hz), 7.88 (s, 1H), 7.56 (d, 1H, J=8.5), 7.21 (s, 1H), 7.08 (d, 1H, J=9.0), 6.78 (d, 1H, J=5.0), 4.20 (t, 2H, J=6.5), 3.66 (t, 2H, J=7.5), 3.12 (s, 3H), 2.96 (t, 2H, J=7.5), 2.34 (s, 3H), 2.20 (m 2H), 1.75 (m, 2H), 1.00 (t, 3H, J=7.5).

MS: m/z=451 (M+H).

### EXAMPLE 41

Preparation of N′-(2-thiazolyl)-N-methyl-N-(3-{[7-propyl-3-(trifluoromethyl)-1,2-benzisoxazol-6-yl]oxy}propyl)urea.

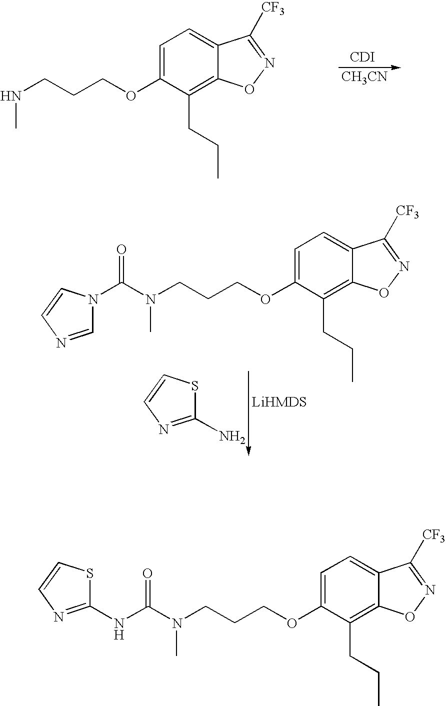

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water ( 2×5 mL) and with brine (5 mL), dried over Na₂SO₄, filtered and the solvent was evaporated in vacuo. At 0° C., LiHMDS (1 M hexanes, 0.29 mL, 0.29 mmol) was added to the indicated thiazolyl amine (18.0 mg, 0.18 mmol) in THF (1 mL) and stirred for 30 min. At 0° C., the mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL) and the combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 7.58 (d, 1H, J=9.0 Hz), 7.33 (s, 1H), 7.06 (d, 1H, J=8.5), 6.88 (s, 1H), 4.17 (t, 2H, J=6.0), 3.67 (t, 2H, J=7.0), 3.09 (s, 3H), 2.95 (t, 2H, J=7.0), 2.19 (m, 2H), 1.75 (m, 2H), 0.99 (t, 3H, J=7.5).

MS: m/z=443 (M+H).

### EXAMPLE 42

Preparation of N′-(5-carbomethoxy-2-pyridyl)-N-methyl-N-(3-{[7-propyl-3-(trifluoromethyl)-1,2-benzisoxazol-6-yl]oxy}propyl)urea.

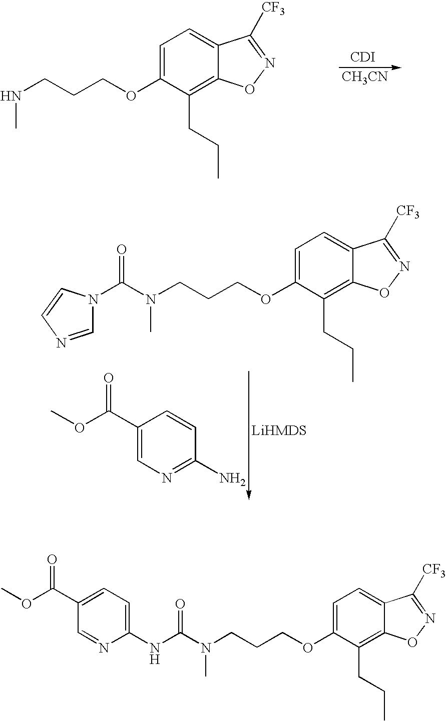

At 0° C., CDI (102.5 mg, 0.63 mmol) was added to the 7-propyl-3-(trifluoromethyl)-6-(3-methylaminopropyloxy)-1,2-benzisoxazole(100.0 mg, 0.31 mmol) in acetonitrile (5 mL), then stirred at RT for 2 hours. The reaction mixture was then poured into ice water (10 mL) and diluted with ethyl acetate (10 mL). The aqueous layer was extracted with ethyl acetate and the combined ethyl acetate extracts were washed with water (2×10 mL) and with brine (10 mL), dried over Na₂SO₄, filtered and the solvent was evaporated in vacuo. At 0° C., LiHMDS (1 M hexanes, 0.95 mL, 0.95 mmol) was added to the indicated amino pyridine (97.0 mg, 0.63 mmol) in THF (2 mL) and stirred for 30 min. At 0° C., the mixture was added to the acyl imidazole, prepared above, in THF (4 mL) and stirred at RT overnight. Another addition of LiHMDS (1 M hexanes, 1.9 mL, 1.9 mmol) was made to the reaction mixture at 0° C. and stirred at RT for 5 hours. NH₄Cl (sat'd aq, 10 mL) was then added into the reaction mixture and diluted with ethyl acetate (10 mL) and the aqueous layer was extracted with ethyl acetate (2×10 mL) and the combined ethyl acetate extracts were washed with H₂O (1×15 mL), NH₄Cl (sat'd aq, 1×10 mL), brine (1×10 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 8.78 (s, 1H), 8.23 (d, 1H, J=8.0 Hz), 8.12 (d, 1H, J=8.0), 7.57 (d, 1H, J=8.5), 7.50 (s, 1H), 7.07 (d, 1H, J=9.0), 4.21 (t, 2H, J=6.0), 3.93 (s, 3H), 3.67 (t, 2H, J=7.0), 3.12 (s, 3H), 2.97 (t, 2H, J=7.0), 2.21 (m 2H), 1.77 (m, 2H), 1.00 (t, 3H, J=7.5).

MS: m/z=495 (M+H).

### EXAMPLE 43

Preparation of N′-(5-carboxy-2-pyridyl)-N-methyl-N-(3-{[7-propyl-3-(trifluoromethyl)-1,2-benzisoxazol-6-yl]oxy}propyl)urea.

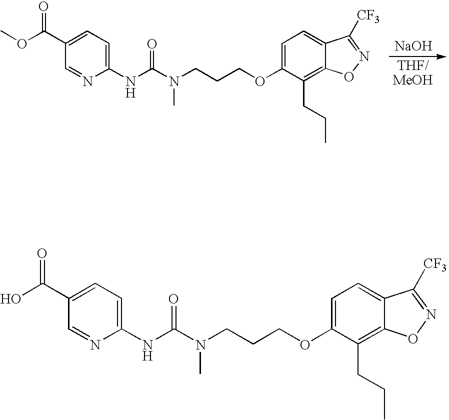

Aqueous NaOH (1 M, 4 mL, 4.0 mmol) was added to the methyl ester prepared in Example 42 (250.0 mg, 0.51 mmol) in THF (5 mL) and MeOH (10 mL), then stirred at RT overnight. The THF was evaporated in vacuo and the residue was extracted with ether (2×30 mL). The mixture was then was neutralized with HCl (1N), and extracted with ethyl acetate (2×30 mL). The combined ethyl acetate layer extracts were washed with water (2×20 mL) and brine (20 mL), dried over Na₂SO₄, filtered and the solvent was evaporated in vacuo. The crude product was triturated with methylene chloride and filtered to give the title compound.

¹H NMR (CDCl₃) δ 9.73 (br, 1H), 8.78 (s, 1H), 8.23 (d, 1H, J=8.0 Hz), 8.12 (d, 1H, J=8.0), 7.57 (d, 1H, J=8.5), 7.07 (d, 1H, J=9.0), 4.21 (t, 2H, J=6.0), 3.67 (t, 2H, J=7.0), 3.12 (s, 3H), 2.97 (t, 2H, J=7.0), 2.21 (m 2H), 1.77 (m, 2H), 1.00 (t, 3H, J=7.5).

MS: m/z=481 (M+H).

### EXAMPLE 44

Preparation of N′-(5-chloro-2-pyridyl)-N-methyl-N-(3-{[7-propyl-3-(trifluoromethyl)-1,2-benzisoxazol-6-yl]oxy}propyl)urea.

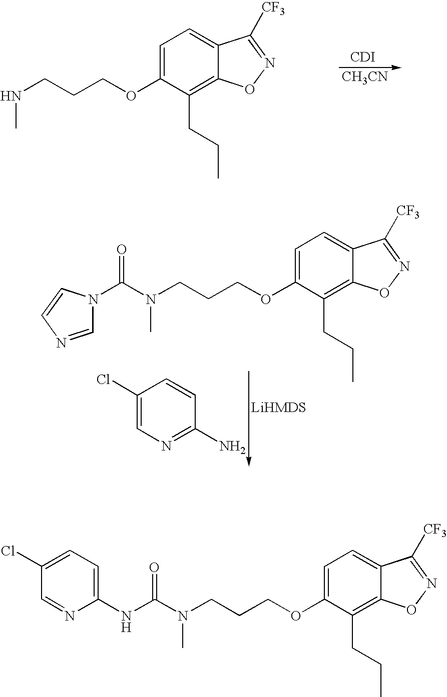

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), dried over Na₂SO₄, filtered and the solvent was evaporated in vacuo. At 0° C., LiHMDS (1 M hexanes, 0.29 mL, 0.29 mmol) was added to the indicated amino pyridine (23.14 mg, 0.18 mmol) in THF (1 mL) and stirred for 30 min. At 0° C., the mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL) and the combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 8.10 (d, 1H, J=2.5 Hz), 8.03 (d, 1H, J=9.0), 7.60 (m, 2H), 7.25(s, 1H), 7.07 (d, 1H, J=9.0), 4.20 (t, 2H, J=6.0), 3.67 (t, 2H, J=7.0), 3.11 (s, 3H), 2.97 (t, 2H, J=7.0), 2.21 (m 2H), 1.77 (m, 2H), 1.00 (t, 3H, J=7.5).

MS: m/z=471.5(M+H).

### EXAMPLE 45

Preparation of N′-(5-methyl-2-pyridyl)-N-methyl-N-(3-{[7-propyl-3-(trifluoromethyl)-1,2-benzisoxazol-6-yl]oxy}propyl)urea.

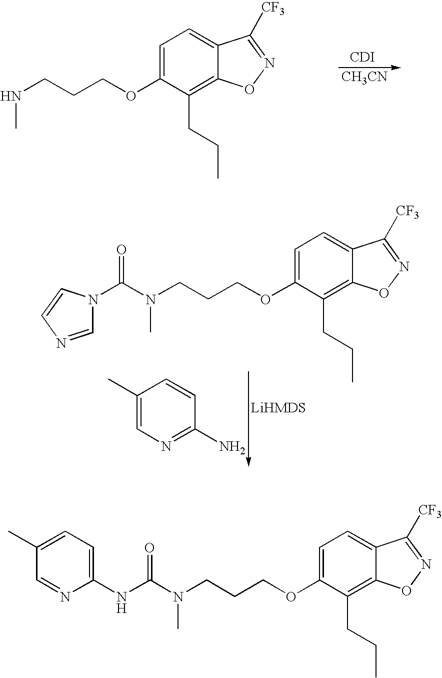

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), dried over Na₂SO₄, filtered and the solvent was evaporated in vacuo. At 0° C., LiHMDS (1 M hexanes, 0.29 mL, 0.29 mmol) was added to the indicated amino pyridine (20.4 mg, 0.18 mmol) in THF (1 mL) and stirred for 30 min. At 0° C., the mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL) and the combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 7.99 (s, 1H), 7.91(d, 1H, J=8.5 Hz), 7.56 (d, 1H, J=8.5), 7.45 (m, 1H), 7.16 (s, 1H), 7.08 (d, 1H, J=9.0), 4.20 (t, 2H, J=6.0), 3.67 (t, 2H, J=7.0), 3.11 (s, 3H), 2.97 (t, 2H, J=7.0), 2.27 (s, 3H), 2.21 (m 2H), 1.77 (m, 2H), 1.00 (t, 3H, J=7.5).

MS: m/z=451(M+H).

### EXAMPLE 46

Preparation of N′-(3-methyl-5-oxazolyl)-N-methyl-N-(3-{[7-propyl-3-(trifluoromethyl)-1,2-benzisoxazol-6-yl]oxy}propyl)urea.

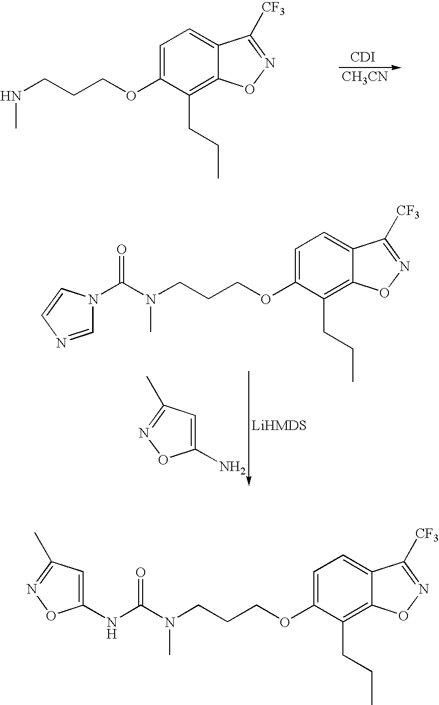

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate extracts were washed with water (2×5 mL) and with brine (5 mL), dried over Na₂SO₄, filtered and the solvent was evaporated in vacuo. At 0° C., LiHMDS (1 M hexanes, 0.29 mL, 0.29 mmol) was added to the indicated oxazolyl amine (17.7 mg, 0.18 mmol) in THF (1 mL) and stirred for 30 min. At 0° C., the mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL) and the combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 7.58 (d, 1H, J=8.5 Hz), 7.45 (s, 1H), 7.08 (d, 1H, J=8.5), 6.03 (s, 1H), 4.19 (t, 2H, J=6.0), 3.64 (t, 2H, J=6.5), 3.10 (s, 3H), 2.97 (t, 2H, J=7.5), 2.24 (s, 3H), 2.19 (m, 2H), 1.76 (m, 2H), 1.00 (t, 3H, J=7.0).

MS: m/z=441 (M+H).

### EXAMPLE 47

Preparation of N′-(5-fluoro-2-pyridyl)-N-methyl-N-(3-{[7-propyl-3-(trifluoromethyl)-1,2-benzisoxazol-6-yl]oxy}propyl)urea.

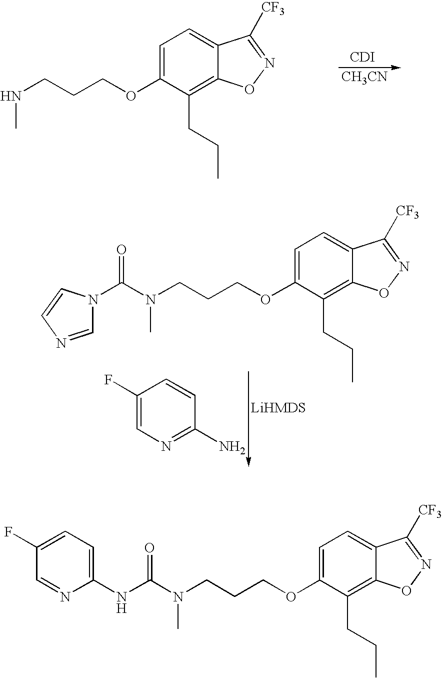

At 0° C., CDI (61.0 mg, 0.377 mmol) was added to the 7-propyl-3-(trifluoromethyl)-6-(3-methylaminopropyloxy)-1,2-benzisoxazole(59.5 mg, 0.188 mmol) in acetonitrile (3 mL), then stirred at RT for 2 hours. The reaction mixture was then poured into ice water (5 mL) and diluted with ethyl acetate (5 mL). The aqueous layer was extracted with ethyl acetate and the combined ethyl acetate solvent was evaporated in vacuo. At 0° C., LiHMDS (1 M hexanes, 0.29 mL, 0.29 mmol) was added to the indicated amino pyridine (20.2 mg, 0.18 mmol) in THF (1 mL) and stirred for 30 min. At 0° C., the mixture was added to the acyl imidazole, prepared above, in THF (2 mL) and stirred at RT overnight. Another addition of LiHMDS (1 M hexanes, 0.19 mL, 0.19 mmol) was made to the reaction mixture at 0° C. and stirred at RT for 5 hours. NH₄Cl (sat'd aq, 5 mL) was then added into the reaction mixture and diluted with ethyl acetate (5 mL) and the aqueous layer was extracted with ethyl acetate (2×5 mL) and the combined ethyl acetate extracts were washed with H₂O (1×15 mL), NH₄Cl (sat'd aq, 1×10 mL), brine (1×5 mL), then dried over Na₂SO₄ and filtered. The solvent was evaporated in vacuo, and the residue was purified by chromatography on silica gel using MeOH:methylene chloride (2:98) to give the title compound.

¹H NMR (CDCl₃) δ 8.04 (m, 2H), 7.55 (d, 1H, J=8.5), 7.39 (m, 1H), 7.23 (s, 1H), 7.08 (d, 1H, J=8.5), 4.20 (t, 2H, J=6.0), 3.67 (t, 2H, J=7.0), 3.11 (s, 3H), 2.97 (t, 2H, J=7.0), 2.21 (m 2H), 1.77 (m, 2H), 1.00 (t, 3H, J=7.5).

MS: m/z=455(M+H).

### EXAMPLE 48

Preparation of N′-(4-carbethoxyphenyl)-N-methyl-N-(3-{[7-propyl-3-(trifluoromethyl)-1,2-benzisoxazol-6-yl]oxy}propyl)urea.

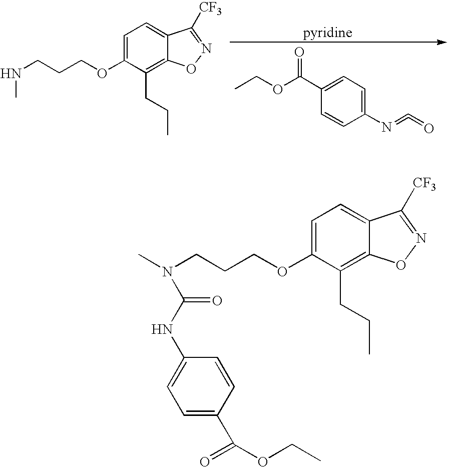

To a solution of the 7-propyl-3-(trifluoromethyl)-6-(3-methylaminopropyloxy)-1,2-benzisoxazole(50.0 mg, 0.16 mmol) in methylene chloride (2 mL) was added ethyl 4-isocyanato benzoate (34.5 mg, 0.17 mmol) and the reaction mixture was stirred at RT for 2 hours. The solvent was evaporated in vacuo, and the residue was purified by chromatography on silica gel using MeOH:methylene chloride (1:49) to give the title compound.

¹H NMR (CDCl₃) δ 7.94 (d, 2H, J=9.0 Hz), 7.59 (d, 1H, J=8.0),7.37 (d, 2H, J=9.0), 7.07 (d, 1H, J=5.5), 6.65 (s, 1H), 4.36 (m 2H), 4.23 (t, 2H, J=6.0), 3.67 (t, 2H, J=7.0), 3.12 (s, 3H), 2.97 (t, 2H, J=7.5), 2.21 (m 2H), 1.77 (m, 2H), 1.40 (t, 3H, J=7.0), 0.99 (t, 3H, J=7.0).

MS: m/z=508 (M+H).

### EXAMPLE 49

Preparation of N′-(4-carboxyphenyl)-N-methyl-N-(3-{[7-propyl-3-(trifluoromethyl)-1,2-benzisoxazol-6-yl]oxy}propyl)urea.

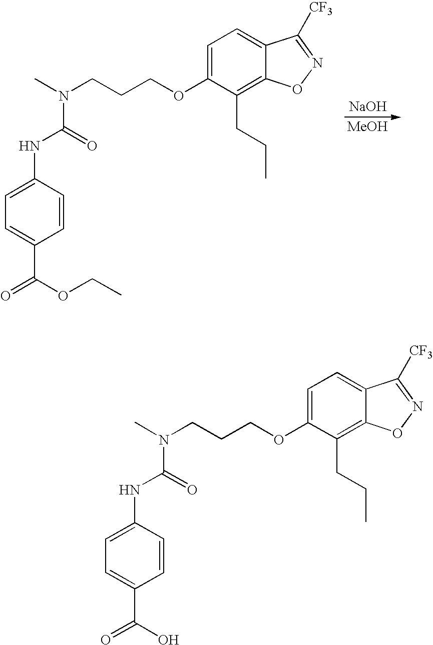

The urea ester starting material prepared according to the procedure of Example 50 (43.0 mg, 0.085 mmol) in MeOH (2 mL) and THF (1 mL) was stirred under N₂ followed by addition of NaOH aq (0.3 mL) and stirring at RT overnight. The reaction mixture was then neutralized with 1N HCl and extracted with ethyl acetate. The combined ethyl acetate extracts were washed with brine, dried over Na₂SO₄ and filtered. The solvent was evaporated in vacuo, and the residue was purified by chromatography on silica gel using ethyl acetate:hexanes (30:70) 2.5% acetic acid to give the title compound.

¹H NMR (CD₃OD) δ 7.84 (d, 2H, J=9.0 Hz), 7.61 (d, 1H, J=8.0), 7.38 (d, 2H, J=9.0), 7.26 (d, 1H, J=5.5), 4.58 (br, 1H), 4.23 (t, 2H, J=6.0), 3.67 (t, 2H, J=7.0), 3.12 (s, 3H), 2.97 (t, 2H, J=7.5), 2.21 (m, 2H), 1.77 (m, 2H), 0.99 (t, 3H, J=7.0).

MS: m/z=480 (M+H).

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound of formula I 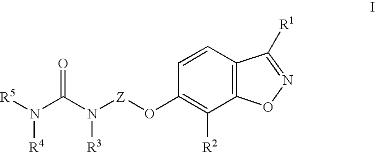
and the pharmaceutically acceptable salts, esters and tautomers thereof, wherein
R¹ is selected from the group consisting of:
(a) —CF₃,
(b) —CH₂C(CH₃)₃,
(c) phenyl, unsubstituted, mono- or poly-substituted with halo,
(d) —C₁₋₆ alkyl, and
(e) —C₁₋₂ alkyl-phenyl;
R² is selected from the group consisting of:
(a) —C₁₋₆ alkyl,
(b) —CO₂R⁶,
(c) —CR⁶R⁷—O—R⁸,
(d) —CR⁶R⁷—S—R⁸, and
(e) —COR⁶;
R³ is —C₁₋₆alkyl;
R⁴ is —H or —C₁₋₆alkyl;
R⁵ is selected from the group consisting of:
(a) —H,
(b) C₁₋₆ alkyl, unsubstituted or monosubstituted with —CO₂R⁶,
(c) phenyl, unsubstituted or monosubstituted with —CO₂R⁶,
(d) tetrazolyl,
(e) oxazolyl, unsubstituted, mono- or polysubstituted with a substituent independently selected at each occurrence from the group consisting of halo, —C₁₋₆alkyl and —CO₂R⁶,
(f) thiazolyl, unsubstituted, mono- or polysubstituted with a substituent independently selected at each occurrence from the group consisting of halo, —C₁₋₄alkyl and —CO₂R⁶,
(g) pyridyl, unsubstituted, mono- or polysubstituted with a substituent independently selected at each occurrence from the group consisting of halo, —C₁₋₆alkyl and O₂R⁶,
(h) pyrimidinyl, unsubstituted, mono- or polysubstituted with a substituent independently selected at each occurrence from the group consisting of halo, —C₁₋₆alkyl and —CO₂R⁶,
(i) pyrazinyl, unsubstituted, mono- or polysubstituted with a substituent independently selected at each occurrence from the group consisting of halo, —C₁₋₆alkyl and —CO₂R⁶, and
(l) N-oxo-pyridyl, unsubstituted, mono- or polysubstituted with a substituent independently selected at each occurrence from the group consisting of halo, —C₁₋₆alkyl and —CO₂R⁶;
R⁶, R⁷ and R⁸ are independently selected at each occurrence from the group consisting of —H, phenyl, and —C₁₋₆ alkyl; and
Z is —C₁₋₆ alkanediyl-.

2. The compound of claim 1 wherein R¹ is —CF₃.

3. The compound of claim 1 wherein R² is —C₁₋₆ alkyl.

4. The compound of claim 1 wherein R³ is C₁₋₆alkyl.

5. The compound of claim 1 wherein R⁴ is —H or methyl.

6. The compound of claim 1 wherein Z is -n-propanediyl-.

7. The compound of claim 1 wherein R¹ is —CF₃; R² is —C₁₋₆ alkyl; R³ is C₁₋₆alkyl; R⁴ is —H or methyl; and Z is -n-propanediyl-.

8. The compound of claim 1 wherein R⁵ is —C₁₋₆ alkyl, unsubstituted, mono- or polysubstituted with CO₂R⁶.

9. The compound of claim 1 wherein R⁵ is selected from pyridyl, N-oxo-pyridyl, pyrimidinyl, pyrazinyl and tetrazolyl wherein each of pyridyl, N-oxo-pyridyl, pyrimidinyl and pyrazinyl may be unsubstituted, mono- or polysubstituted with a substituent independently selected at each occurrence from the group consisting of halo, —C₁₋₆alkyl and —CO₂R⁶.

10. The compound of claim 1 wherein R⁵ is selected from oxazolyl and thiazoly, unsubstituted, mono- or polysubstituted with a substituent independently selected at each occurrence from the group consisting of —C₁₋₆alkyl and —CO₂R⁶.

11. The compound of claim 1 wherein R⁵ is phenyl, unsubstituted or monosubstituted with —CO₂R⁶.

12. The compound of claim 1 wherein R⁵ is —H.

13. The compound of claim 1 selected from:
(1) N,N′-dimethyl-N-(3-{[7-propyl-3-(trifluoromethyl)-1,2-benzisoxazol-6- yl]oxy}propyl)urea;
(2) N′-ethyl-N-methyl-N-(3-{[7-propyl-3-(trifluoromethyl)-1,2-benzisoxazol-6-yl]oxy}propyl)urea;
(3) N′-propyl-N-methyl-N-(3-{[7-propyl-3-(trifluoromethyl)-1,2-benzisoxazol-6-yl]oxy}propyl)urea;
(4) N′-isopropyl-N-methyl-N-(3-{[7-propyl-3-(trifluoromethyl)-1,2-benzisoxazol-6-yl]oxy}propyl)urea;
(5) N′-(2-carbethoxyethyl)-N-methyl-N-(3-{[7-propyl-3-(trifluoromethyl)-1,2-benzisoxazol-6-yl]oxy}propyl)urea;
(6) N′-methyl-N-ethyl-N-(3-{[7-propyl-3-(trifluoromethyl)-1,2-benzisoxazol-6-yl]oxy}propyl)urea;
(7) N′,N-diethyl-N-(3-{[7-propyl-3-(trifluoromethyl)-1,2-benzisoxazol-6-yl]oxy}propyl)urea;
(8) N′-propyl-N-ethyl-N-(3-{[7-propyl-3-(trifluoromethyl)-1,2-benzisoxazol-6-yl]oxy}propyl)urea;
(9) N′-isopropyl-N-ethyl-N-(3-{[7-propyl-3-(trifluoromethyl)-1,2-benzisoxazol-6-yl]oxy}propyl)urea;
(10) N′-(2-carboxyethyl)-N-ethyl-N-(3-{[7-propyl-3-(trifluoromethyl)-1,2-benzisoxazol-6-yl]oxy}propyl)urea;
(11) N′-carboxymethyl-N-ethyl-N-(3-{[7-propyl-3-(trifluoromethyl)-1,2-benzisoxazol-6-yl]oxy}propyl)urea;
(12) N′-(1(S)-carboxyethyl)-N-methyl-N-(3-{[7-propyl-3-(trifluoromethyl)-1,2-benzisoxazol-6-yl]oxy}propyl)urea;
(13) rac-N′-methyl-N′-(1-carboxyethyl)-N-methyl-N-(3-{[7-propyl-3-(trifluoromethyl)-1,2-benzisoxazol-6-yl]oxy}propyl)urea;
(14) rac-N′-(1-carboxyethyl)-N-methyl-N-(3-{[7-propyl-3-(trifluoromethyl)-1,2-benzisoxazol-6-yl]oxy}propyl)urea;
(15) rac-N′-(2-carboxypropyl)-N-methyl-N-(3-{[7-propyl-3-(trifluoromethyl)-1,2-benzisoxazol-6-yl]oxy}propyl)urea;
(16) N′-ethyl-N′-methyl-N-methyl-N-(3-{[7-propyl-3-(trifluoromethyl)-1,2-benzisoxazol-6-yl]oxy}propyl)urea;
(17) N′,N′-dimethyl-N-methyl-N-(3-{[7-propyl-3-(trifluoromethyl)-1,2-benzisoxazol-6-yl]oxy}propyl)urea;
(18) N′-(2-pyridyl)-N-methyl-N-(3-{[7-propyl-3-(trifluoromethyl)-1,2-benzisoxazol-6-yl]oxy}propyl)urea;
(19) N′-(3-pyridyl)-N-methyl-N-(3-{[7-propyl-3-(trifluoromethyl)-1,2-benzisoxazol-6-yl]oxy}propyl)urea;
(20) N′-(4-pyridyl)-N-methyl-N-(3-{[7-propyl-3-(trifluoromethyl)-1,2-benzisoxazol-6-yl]oxy}propyl)urea;
(21) N′-(2-pyridyl)-N′-methyl-N-methyl-N-(3-{[7-propyl-3-(trifluoromethyl)-1,2-benzisoxazol-6-yl]oxy}propyl)urea;
(22) N′-methyl-N′-(4-pyridyl)-N-methyl-N-(3-{[7-propyl-3-(trifluoromethyl)-1,2-benzisoxazol-6-yl]oxy}propyl)urea;
(23) N′-(6-methyl-2-pyridyl)-N-methyl-N-(3-{[7-propyl-3-(trifluoromethyl)-1,2-benzisoxazol-6-yl]oxy}propyl)urea;
(24) N′-(4,6-dimethyl-2-pyridyl)-N-methyl-N-(3-{[7-propyl-3-(trifluoromethyl)-1,2-benzisoxazol-6-yl]oxy}propyl)urea;
(25) N′-(4-methyl-2-pyridyl)-N-methyl-N-(3-{[7-propyl-3-(trifluoromethyl)-1,2-benzisoxazol-6-yl]oxy}propyl)urea;
(26) N′-(5-chloro-2-pyridyl)-N-methyl-N-(3-{[7-propyl-3-(trifluoromethyl)-1,2-benzisoxazol-6-yl]oxy}propyl)urea;
(27) N′-(5-methyl-2-pyridyl)-N-methyl-N-(3-{[7-propyl-3-(trifluoromethyl)-1,2-benzisoxazol-6-yl]oxy}propyl)urea;
(28) N′-(5-fluoro-2-pyridyl)-N-methyl-N-(3-{[7-propyl-3-(trifluoromethyl)-1,2-benzisoxazol-6-yl]oxy}propyl)urea;
(29) N′-(5-carbomethoxy-2-pyridyl)-N-methyl-N-(3-{[7-propyl-3-(trifluoromethyl)-1,2-benzisoxazol-6-yl]oxy}propyl)urea;
(30) N′-(5-carboxy-2-pyridyl)-N-methyl-N-(3-{[7-propyl-3-(trifluoromethyl)-1,2-benzisoxazol-6-yl]oxy}propyl)urea;
(31) N′-(1-oxo-2-pyridyl)-N-methyl-N-(3-{[7-propyl-3-(trifluoromethyl)-1,2-benzisoxazol-6-yl]oxy}propyl)urea;
(32) N′-(2-pyrimidinyl)-N-methyl-N-(3-{[7-propyl-3-(trifluoromethyl)-1,2-benzisoxazol-6-yl]oxy}propyl)urea;
(33) N′-(4-pyrimidinyl)-N-methyl-N-(3-{[7-propyl-3-(trifluoromethyl)-1,2-benzisoxazol-6-yl]oxy}propyl)urea;
(34) N′-(2-pyrazinyl)-N-methyl-N-(3-{[7-propyl-3-(trifluoromethyl)-1,2-benzisoxazol-6-yl]oxy}propyl)urea;
(35) N′-(5-tetrazolyl)-N-methyl-N-(3-{[7-propyl-3-(trifluoromethyl)-1,2-benzisoxazol-6-yl]oxy}propyl)urea; and the pharmaceutically acceptable salts, esters and tautomers thereof.
(36) N′-(5-methyl-3-oxazolyl)-N-methyl-N-(3-{[7-propyl-3-(trifluoromethyl)-1,2-benzisoxazol-6-yl]oxy}propyl)urea;
(37) N′-(2-thiazolyl)-N-methyl-N-(3-{[7-propyl-3-(trifluoromethyl)-1,2-benzisoxazol-6-yl]oxy}propyl)urea;
(38) N′-(3-methyl-5-oxazolyl)-N-methyl-N-(3-{[7-propyl-3-(trifluoromethyl)-1,2-benzisoxazol-6-yl]oxy}propyl)urea; and the pharmaceutically acceptable salts, esters and tautomers thereof.
(39) N′-phenyl-N-methyl-N-(3-{[7-propyl-3-(trifluoromethyl)-1,2-benzisoxazol-6-yl]oxy}propyl)urea;
(40) N′-phenyl-N-ethyl-N-(3-{[7-propyl-3-(trifluoromethyl)-1,2-benzisoxazol-6-yl]oxy}propyl)urea;
(41) N′-(4-carbethoxyphenyl)-N-methyl-N-(3-{[7-propyl-3-(trifluoromethyl)-1,2-benzisoxazol-6-yl]oxy}propyl)urea;
(42) N′-(4-carboxyphenyl)-N-methyl-N-(3-{[7-propyl-3-(trifluoromethyl)-1,2-benzisoxazol-6-yl]oxy}propyl)urea; and
(43) N-methyl-N-(3-{[7-propyl-3-(trifluoromethyl)-1,2-benzisoxazol-6-yl]oxy}propyl)urea;
and the pharmaceutically acceptable salts, esters and tautomers thereof.

14. A method for treating depressed plasma HDL cholesterol levels comprising administering a therapeutically effective amount of a compound of claim 1 to a patient in need thereof.

15. A method for reducing the risk of occurrence of atherosclerosis or an atherosclerotic disease event comprising administering a prophylactically effective amount of a compound of claim 1 to a patient at risk for atherosclerosis or an atherosclerotic disease event.

16. A method for treating atherosclerosis comprising administering a therapeutically effective amount of a compound of claim 1 to a patient who has atherosclerotic disease.

17. A pharmaceutical composition comprising a compound of claim 1 and a pharmaceutically acceptable carrier.

18. A pharmaceutical composition made by combining a compound of claim 1 10 with a pharmaceutically acceptable carrier.

19. A process for preparing a pharmaceutical composition comprising combining a compound of claim 1 with a pharmaceutically acceptable carrier.
